(19) United States
(12) Patent Application Publication
(10) Pub. No.: US 2026/0315630 A1
(43) Pub. Date: Oct. 15, 2026

(54) RADIOLABELLED COMPOUND OF A QUATERNARY AMMONIUM SALT OF A POLYCYCLIC AROMATIC AMINE AND METHODS OF MANUFACTURING AND DIAGNOSTIC USE THEREOF

(71) Applicant: SYNEKTIK S.A., Warszawa (PL)

(72) Inventors: Joanna Towpik, Warszawa (PL); Seweryn Andrzej Krajewski, Warszawa (PL); Joanna Wlostowska, Warszawa (PL); Lukasz Marek Steczek, Warszawa (PL)

(73) Assignee: SYNEKTIK S.A., Warszawa (PL)

(21) Appl. No.: 19/312,604

(22) Filed: Aug. 28, 2025

Related U.S. Application Data
(63) Continuation of application No. 17/273,647, filed on Mar. 4, 2021.

(30) Foreign Application Priority Data
Sep. 5, 2018 (PL) P.426916
Sep. 4, 2019 (WO) PCT/PL2019/000073

(51) Int. Cl. A61K 51/04; C07B 59/00; C07D 215/12; C07D 215/227; C07D 219/02; C07D 219/06; C07D 219/08

(52) U.S. Cl. CPC A61K 51/0455; C07B 59/002; C07D 215/12; C07D 215/227; C07D 219/02; C07D 219/06; C07D 219/08; C07B 2200/05

(57) ABSTRACT

The disclosure relates to a radioisotope-labelled compound having a structure according to formula I, wherein a wavy line indicates a single bond between a non-nodal carbon atom of a polycyclic aromatic system and an R¹ substituent selected from a hydrogen; a halogen; a hydroxy; a protected hydroxy; a C₁₋₄ alkoxy; a nitro group; an amino group; an amino group having 1 hydrogen replaced with a C₁-C₆ alkyl group; an amino group having 2 hydrogens replaced with a C₁-C₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl; wherein R² is a chain aliphatic substituent having: a total of 1-16 carbon atoms, an atom of ¹⁸F fluorine radioisotope replacing a hydrogen atom at one of the carbon atoms, and a —CH₂ fragment as a terminal member of a chain, wherein the chain connects to one of a hydrogen, a phenyl group, and a phenyl group having 1-3 substituents selected from halogens and C₁₋₆ alkyl, and wherein if the chain contains at least 2 carbon atoms and there is a bivalent link between the chain carbon atoms, then the bivalent link is selected from the group consisting of an oxygen atom —O—, a sulfur atom —S—, and a C₃₋₆ cycloalkylene; wherein R³ and R⁴ are combined to form a bivalent butadienyl-1,3 substituent whose terminal carbon atoms are linked to adjacent non-nodal carbon atoms of a B ring to form an aromatic C ring fused with an A and B ring system, having R¹ substituents at non-nodal carbon atoms; wherein n is an integer of 9; wherein X⁻ is a pharmaceutically acceptable counter ion selected from: an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

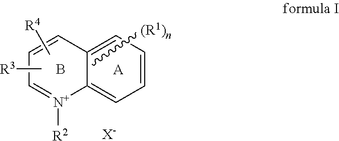

### CROSS REFERENCE TO RELATED APPLICATIONS

This application is a continuation of U.S. application Ser. No. 17/273,647, which is a US national stage application under 35 USC § 371 of PCT/PL2019/000073 filed on Sep. 4, 2019, which claims the benefit of PL Application No. P.426916 filed Sep. 5, 2018, which are incorporated by reference herein in their entirety.

### FIELD OF THE DISCLOSURE

The disclosure relates to a radioisotope-labelled compound of quaternary ammonium salt of a polycyclic aromatic amine, the use of said compound in a diagnostic method of positron emission tomography, and a pharmaceutical composition containing the radioisotope-labelled compound of a polycyclic quaternary aromatic amine.

### BACKGROUND

Nuclear cardiology based on non-invasive imaging studies using radiopharmaceuticals (radiolabelled molecules) makes it possible to assess the function of the cardiovascular system in a safe, fast and relatively non-expensive fashion. Nuclear cardiology procedures are included in various guidelines for the diagnosis and therapy of coronary artery disease. Hybrid PET-CT (positron emission tomography combined with computed tomography) and SPECT-CT (single photon emission computed tomography combined with computed tomography) methods are two well-established, non-invasive imaging techniques used for cardiological diagnostics.

SPECT-CT is an important, non-invasive, widespread method for imaging myocardial perfusion that provides information on all of the myocardial viability, perfusion and function. To assess perfusion, compounds labelled with technetium-99m (sestamibi and tetrophosmin) and thallium-201 (thallium chloride-201) are used. They are comparable in terms of efficiency in detecting coronary artery disease.

PET-CT is used to evaluate the metabolism of glucose, oxygen, perfusion and receptor function. The PET-CT study is useful in diagnosing myocardial viability, and, in addition, it allows for measuring the myocardial physiological activity. The PET-CT study uses radiopharmaceuticals labelled with positron-emitting radionuclides, such as unstable isotopes of elements essential in the structure of living organisms: oxygen (O¹⁵), nitrogen (¹³N) and carbon (¹¹C).

The usability of compounds labelled with said radionuclides is limited to short T_{1/2} half-life times of 2 min, 10 min and 20 min for ¹⁵O, ¹³N and ¹¹C, respectively. Another positron-emitting radionuclide is the ¹⁸F fluorine isotope; therefore, ¹⁸F-labelled compounds were also used in PET-CT studies. ¹⁸F-fluorodeoxyglucose (¹⁸F-FDG), which, upon bodily administration, is involved in the metabolic pathway of glucose, is the most common compound used in clinical practice. The ¹⁸F-FDG study allows for monitoring myocardial metabolic changes in patients with chronic heart failure, so that it provides an excellent prognostic tool in chronic heart failure. Since the half-life T_{1/2} of the ¹⁸F radioisotope is longer than that of ¹⁵O, ¹³N or ¹¹C (T_{1/2} for ¹⁸F is 109.8 min), the ¹⁸F-labelled radiopharmaceutical may be manufactured in locations other than the study performed.

International publication WO 2011/084585 discloses an disclosure of a radiolabelled dihydroethidine derivative having a triphenylphosphonioalkylene group attached to the nitrogen atom of the heterocyclic ring and a phenyl group attached to the sp³ carbon atom of said heterocyclic ring. The phenyl group is substituted by other substituents in which a fluorine atom may be present. According to one of the aspects of the solution, the radioisotope is an isotope emitting positron radiation, especially ¹¹C or ¹⁸F. The compound according to the disclosure is designed for visualising the distribution of free oxygen radicals, especially peroxide anion radicals in an animal body, since dihydroethidine derivatives are oxidised by peroxides, and the use of positron emission tomography imaging provides detailed data on the distribution of peroxide anion radicals in vivo.

Methods are known for manufacturing selected quaternary aromatic amines containing atoms of ¹⁹F fluorine (i.e. non-radioactive fluorine isotope) in aliphatic substituent of the nitrogen atom of the amine. U.S. Pat. No. 4,062,849 discloses the manufacturing of N-heptadecafluorodecyl acridinium iodide using N-heptadecafluorodecyl iodide (C₈F₁₇C₂H₄I) and a corresponding aromatic amine. Moreover, a scientific article entitled “An unusual substitution reaction directed by an intramolecular re-arrangement” (A. D. C. Parenty, L. V. Smith, L. Cronin), Tetrahedron, 61 (2005), pp. 8410-8418, discloses the manufacturing of 2-fluoroethyl phenanthridinium bromide from 2-fluoroethyl tosylate and phenanthridine with the subsequent exchange of counterion for bromide anion using Dowex 1X-850 resin pre-treated with saturated sodium bromide solution.

### SUMMARY

The present disclosure relates, according to some embodiments, to a radioisotope-labelled compound comprising a quaternary ammonium salt of a polycyclic aromatic amine having a structure according to formula I,

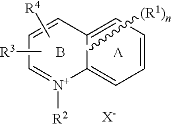

A wavy line may indicate a single bond between a non-nodal carbon atom of a polycyclic aromatic system and an R¹ substituent. An R¹ substituent may be selected from a hydrogen; a halogen; a hydroxy; a protected hydroxy; a C₁₋₄ alkoxy; a nitro group; an amino group; an amino group having 1 hydrogen replaced with a C₁-C₆ alkyl group; an amino group having 2 hydrogens replaced with a C₁-C₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl. An R² may include a chain aliphatic substituent having a total of 1-16 carbon atoms, an atom of ¹⁸F fluorine radioisotope replacing a hydrogen atom at one of the carbon atoms, and a —CH₂ fragment as a terminal member of a chain. A chain may connect to one of a hydrogen, a phenyl group, and a phenyl group having 1-3 substituents selected from halogens and C₁₋₆ alkyl. In some embodiments, if a chain contains at least 2 carbon atoms and there is a bivalent link between the chain carbon atoms, then the bivalent link may be selected from the group consisting of an oxygen atom —O—, a sulfur atom —S—, and a C₃₋₆ cycloalkylene. An R³ and an R⁴ may be combined to form a bivalent butadienyl-1,3 substituent whose terminal carbon atoms are linked to adjacent non-nodal carbon atoms of a B ring to form an aromatic C ring fused with an A and B ring system, while having R¹ substituents at non-nodal carbon atoms. An n may be an integer of 9. An X⁻ may be a pharmaceutically acceptable counter ion selected from an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

An R¹ substituent may be selected from a hydrogen; a halogen; a C₁₋₄ alkoxy; a nitro group; an amino group; an amino group having 1 hydrogen replaced with a C₁-C₄ alkyl group; an amino group having 2 hydrogens replaced with the C₁-C₄ alkyl group; a chain C₁₋₄ carbon group; a chain C₁₋₄ carbon group having a substituent selected from a halogen and a C₁₋₄ alkanesulfonic; a phenyl group; a phenyl group having 1-3 substituents independently selected from halogens, a chain C₁₋₄ carbon, a halogenated chain C₁₋₄ carbon substituent, a C₁₋₄ alkoxy, and an amino group having 1-2 atoms of hydrogen replaced a C₁₋₄ alkyl. An R², if a chain contains at least 2 carbon atoms and there is a bivalent link between the chain carbon atoms, then the link may be selected from the group consisting of an oxygen atom —O— and a sulfur atom —S—.

An R¹ substituent may be selected from a hydrogen; a halogen; a C₁₋₄ alkoxy; an amino group; an amino group having 1 hydrogen replaced with a C₁-C₄ alkyl group; an amino group having 2 hydrogens replaced with the C₁-C₄ alkyl group; a chain C₁₋₄ carbon group; a chain C₁₋₄ carbon group having a substituent selected from a halogen, a phenyl group; a phenyl group having 1-3 substituents independently selected from halogens, a chain C₁₋₄ carbon, a halogenated chain C₁₋₄ carbon substituent, and a C₁₋₄ alkoxy. An R², if a chain contains at least 2 carbon atoms and there is a bivalent link between a chain carbon atoms, then the link is an oxygen atom —O—.

An R¹ substituent may be selected from a hydrogen; a halogen; a C₁₋₄ alkoxy; an amino group; an amino group having 1 hydrogen replaced with a C₁-C₂ alkyl group; an amino group having 2 hydrogens replaced with a C₁-C₂ alkyl group; a chain C₁₋₄ carbon group; a chain C₁₋₄ carbon group having a substituent selected from a halogen, a phenyl group; a phenyl group having 1-3 substituents independently selected from halogens, a chain C₁₋₄ carbon, and a halogenated chain C₁₋₄ carbon substituent. For R², if a bivalent link between a chain carbon atoms is absent, then a chain may connect to one of a hydrogen, a phenyl group, and a phenyl group having 1-3 substituents selected from halogens and C₁₋₄ alkyl.

An R¹ substituent is selected from a hydrogen; a halogen; an amino group; an amino group having 1 hydrogen replaced with a C₁-C₂ alkyl group; an amino group having 2 hydrogens replaced with a C₁-C₂ alkyl group; a chain C₁₋₄ carbon group; a phenyl group; a phenyl group having 1-3 substituents independently selected from halogens, a chain C₁₋₄ carbon, and a halogenated chain C₁₋₄ carbon substituent. For R², a chain may connect to one of a hydrogen, a phenyl group, and a phenyl group having 1-3 substituents selected from halogens.

According to some embodiments, the present disclosure relates to a positron emission tomography diagnostic method. A method may include (a) administering a radioisotope-labelled compound to a subject; and (b) performing a positron emission tomography scan on the subject. A subject may include a mammal. A positron emission tomography diagnostic method may include examining a cardiovascular system of a subject. A method may include testing a myocardial perfusion of a myocardium of a subject to quantify a regional blood flow. A method may include quantifying a coronary reserve of the subject.

In some embodiments, a pharmaceutical composition may include a pharmaceutically acceptable carrier or diluent; and a radioisotope-labelled compound comprising a quaternary ammonium salt of a polycyclic aromatic amine having a structure according to formula I. A pharmaceutical composition may be formulated as a sterile solution.

The present disclosure relates to a method for manufacturing a pharmaceutical composition, the method including combining a pharmaceutically acceptable carrier with a radioisotope-labelled compound, wherein the radioisotope-labelled compound having a quaternary ammonium salt of a polycyclic aromatic amine having a structure according to formula I. A method may include sterilizing a pharmaceutical composition to form a sterile solution.

### DETAILED DESCRIPTION

The object of the disclosure is to provide a radioisotope-labelled compound of quaternary ammonium salt of a polycyclic aromatic amine, the use of the radioisotope-labelled compound of quaternary ammonium salt of a polycyclic aromatic amine in a diagnostic method of positron emission tomography, and to provide a pharmaceutical composition containing said radioisotope-labelled compound of quaternary ammonium salt of a polycyclic aromatic amine.

The disclosure relates to a radioisotope-labelled compound of quaternary ammonium salt of a polycyclic aromatic amine with formula I,

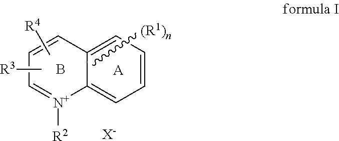

in which formula I
the wavy line indicates a single bond between the non-nodal carbon atom of a polycyclic aromatic system and the R¹ substituent,
R¹ is a substituent independently selected from hydrogen, halogens, a hydroxy optionally protected, C₁₋₄ alkoxy, a nitro group, an amino group optionally having 1 or 2 hydrogen atoms replaced with C₁₋₆ alkyl or having 2 hydrogen atoms replaced with C₂₋₅ alkylene to form a heterocyclic ring, a chain C₁₋₆ carbon group optionally having a halogen, carboxyl, formyl or C₁₋₄ alkanesulfonic substituent, and a phenyl group optionally having 1-5 substituents independently selected from halogens, a chain C₁₋₆ carbon substituent, a halogenated chain C₁₋₆ carbon substituent, a hydroxy optionally protected, C₁₋₄ alkoxy, an amino group optionally having 1-2 hydrogen atoms replaced with C₁₋₆ alkyl,
R² is a chain aliphatic substituent having the —CH₂— fragment as the terminal member of the chain, to which chain a phenyl group is optionally attached optionally having 1-3 substituents selected from halogens and C₁₋₆ alkyl, and, if the chain contains at least 2 carbon atoms, in which chain there is optionally a bivalent link between the chain carbon atoms, said link selected from the oxygen atom —O—, sulfur atom —S— and C₃₋₆ cycloalkylene, wherein the R² substituent contains a total of 1-16 carbon atoms, and the hydrogen atom at one of the carbon atoms is replaced with an atom of ¹⁸F fluorine radioisotope,
R³ and R⁴ are combined to form a bivalent butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms, n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
or a hydrate or solvate thereof.

Preferably, the disclosure relates to a compound with formula I, wherein the wavy line indicates a single bond between the non-nodal carbon atom of a polycyclic aromatic system and the R¹ substituent,
R¹ is a substituent independently selected from hydrogen, halogens, C₁₋₄ alkoxy, a nitro group, an amino group optionally having 1 or 2 hydrogen atoms replaced with C₁₋₄ alkyl, a chain C₁₋₆ carbon group optionally having a halogen or C₁₋₄ alkanesulfonic substituent, and a phenyl group optionally having 1-3 substituents independently selected from halogens, a chain C₁₋₄ carbon substituent, a halogenated chain C₁₋₄ carbon substituent, C₁₋₄ alkoxy, an amino group optionally having 1-2 atoms of hydrogen replaced with C₁₋₄ alkyl,
R² is a chain aliphatic substituent having the —CH₂— fragment as the terminal member of the chain, to which chain a phenyl group is optionally attached optionally having 1-3 substituents selected from halogens and C₁₋₆ alkyl, and, if the chain contains at least 2 carbon atoms, in which chain there is optionally a bivalent link between the carbon atoms, said link selected from the oxygen atom —O— and sulfur atom —S—, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
or a hydrate or solvate thereof.

The disclosure relates in particular to a compound with formula I, wherein the wavy line indicates a single bond between the non-nodal carbon atom of a polycyclic aromatic system and the R¹ substituent,
R¹ is a substituent independently selected from hydrogen, halogens, C₁₋₄ alkoxy, an amino group optionally having 1-2 hydrogen atoms replaced with C₁₋₄ alkyl, a chain C₁₋₄ carbon group optionally having a halogen substituent, and a phenyl group optionally having 1-3 substituents independently selected from halogens, a chain C₁₋₄ carbon substituent, a halogenated chain C₁₋₄ carbon substituent, C₁₋₄ alkoxy,
R² is a chain aliphatic substituent having the —CH₂— fragment as the terminal member of the chain, to which chain a phenyl group is optionally attached optionally having 1-3 substituents selected from halogens and C₁₋₆ alkyl, and, if the chain contains at least 2 carbon atoms, in which chain there is optionally a bivalent link between the carbon atoms, said link being the oxygen atom —O—, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms, n is an integer of 9, X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
or a hydrate or solvate.

The disclosure relates especially to a compound with formula I, wherein the wavy line indicates a single bond between the non-nodal carbon atom of a polycyclic aromatic system and the R¹ substituent,
R¹ is a substituent independently selected from hydrogen, halogens, C₁₋₄ alkoxy, an amino group optionally having 1-2 hydrogen atoms replaced with C₁₋₂ alkyl, a chain C₁₋₄ carbon group optionally having a halogen substituent, and a phenyl group optionally having 1-3 substituents independently selected from halogens, a chain C₁₋₄ carbon substituent and a halogenated chain C₁₋₄ carbon substituent,
R² is a chain aliphatic substituent having the —CH₂— fragment as the terminal member of the chain, to which chain a phenyl group is optionally attached, optionally having 1-3 substituents selected from halogens and C₁₋₄ alkyl, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
or a hydrate or solvate thereof.

Particularly preferably, the disclosure relates to a compound with formula I, wherein the wavy line indicates a single bond between the non-nodal carbon atom of a polycyclic aromatic system and the R¹ substituent,
R¹ is a substituent independently selected from hydrogen, halogens, an amino group optionally having 1-2 hydrogen atoms replaced with C₁₋₂ alkyl, a chain C₁₋₄ carbon group, and a phenyl group optionally having 1-3 substituents independently selected from a chain C₁₋₄ carbon substituent and a halogenated chain C₁₋₄ carbon substituent,
R² is a chain aliphatic substituent having the —CH₂— fragment as the terminal member of the chain, to which chain a phenyl group is optionally attached optionally having a halogen substituent, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
or a hydrate or solvate thereof.

The disclosure relates to a radioisotope-labelled compound as defined above for use in a diagnostic method of positron emission tomography. Preferably, said diagnostic method is used for the examination of the cardiovascular system in a mammal. Said diagnostic method includes a myocardial perfusion imaging for quantifying regional blood flow through the myocardium and/or a cardiac perfusion test to quantify the coronary reserve in coronary artery disease.

The disclosure also relates to a pharmaceutical composition containing a radioisotope-labelled compound specified above and a pharmaceutically acceptable carrier or diluent. Preferably, said composition is in the form of a sterile solution.

The radioisotope-labelled compound is used for the manufacture of an agent for use in a diagnostic method of positron emission tomography. Preferably, the diagnostic method is used for the examination of the cardiovascular system in a mammal. The diagnostic method is used, in particular, for testing cardiac perfusion for quantifying regional blood flow through the myocardium and/or for quantifying the coronary reserve in coronary artery disease.

The disclosure provides a ¹⁸F-radiolabelled compound for use as a cardiotracer to evaluate nocardial perfusion and diagnose coronary artery disease during a PET scan Use of a cardiotracer containing the ¹⁸F radionuclide, for which the range of positrons of the order of 0.6 mm, allows for obtaining a higher spatial resolution of images acquired during the scan and a higher counting sensitivity compared to other PET tracers, such as ⁸²Rb, where the range of positrons is 5.9 mm or ¹³NH₃ (1.6 mm), since the spatial resolution increases as the kinetic energy of positrons decreases. The use of PIT technology and of the cardiotracer according to the disclosure allows for quantifying myocardial blood flow in absolute values including the estimation of the flow through each coronary artery, without the need for invasive catheterisation thereof. Where cardiac perfusion in a PET test is assessed using tracers containing short half-life radionuclides, cardiac perfusion imaging can only be performed in a PET lab with direct access to a cyclotron or generator, since the tracer activity would usually undergo total decay during the time needed for transport to a remote PET lab. Delivery of a cardiotracer in the form of the compound according to the disclosure labelled with the ¹⁸F radionuclide, with a half-life of 109.8 min, allows for producing a cardiotracer outside the PET laboratory and, if necessary, for delivering the pharmaceutical form prepared to the place of testing.

Effective and readily accessible cardiological diagnostics using PET is paramount, because, in addition to contributing to understanding the pathophysiology of heart failure, it provides a tool to assess the outcome of pharmacological and invasive treatment. Apart from the data on organ function (perfusion, metabolism and left ventricular function), the PET-CT method provides quantitative data on the significance of anatomical stenoses in coronary arteries. Both tests, when performed simultaneously, provide a comprehensive diagnostic and prognostic method for the evaluation of patients with chronic heart failure of ischemic aetiology.

The disclosure is described in detail with reference to the drawing, in which

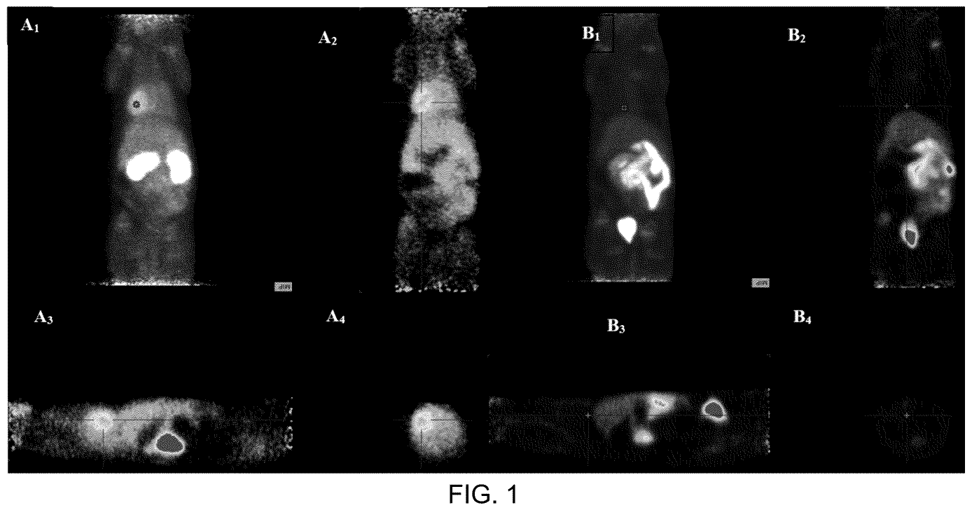

FIG. 1 shows representative PET images of radioactivity distribution obtained in the study on rats following the administration of the pharmaceutical form of 5-(2-[¹⁸F]fluoroethyl)phenanthridinium salt,

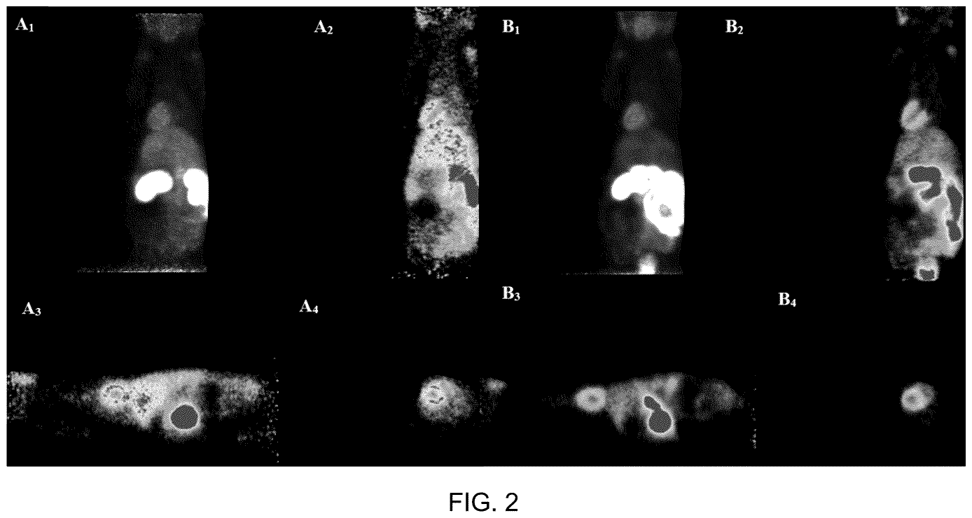

FIG. 2 shows representative PET images of radioactivity distribution obtained in the study on rats following the administration of the pharmaceutical form of 6-phenyl-5-(2-[¹⁸F]fluoroethyl)phenanthridinium salt, and

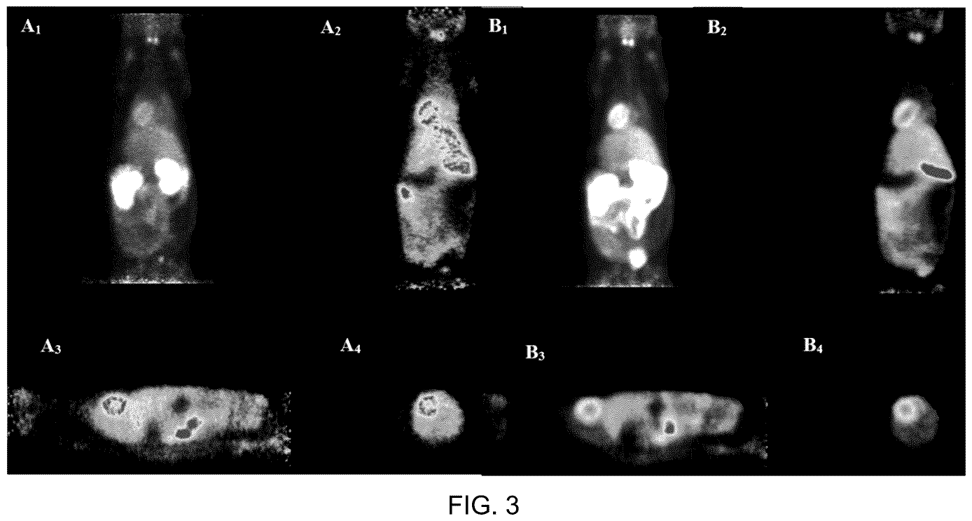

FIG. 3 shows representative PET images obtained in the study on rats following the administration of the pharmaceutical form of 3,6-bis(dimethylamino)-10-(2-[¹⁸F]fluoroethyl)acridinium salt.

The disclosure relates to a radioisotope-labelled compound of quaternary ammonium salt of a polycyclic aromatic amine with formula I,

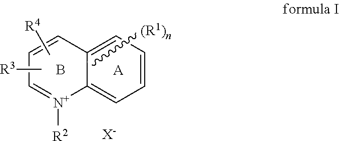

in which formula I
the wavy line indicates a single bond between the non-nodal carbon atom of a polycyclic aromatic system and the R¹ substituent,
R¹ is a substituent independently selected from hydrogen, halogens, a hydroxy optionally protected, C₁₋₄alkoxy, a nitro group, an amino group optionally having 1 or 2 hydrogen atoms replaced with C₁₋₆ alkyl or having 2 hydrogen atoms replaced with C₂₋₅ alkylene to form a heterocyclic ring, a chain C₁₋₆ carbon group optionally having a halogen, carboxy, formyl or C₁₋₄ alkanesulfonic substituent, and a phenyl group optionally having 1-5 substituents independently selected from halogens, a chain C₁₋₆ carbon, a halogenated chain C₁₋₆ carbon substituent, a hydroxy optionally protected, C₁₋₄ alkoxy, an amino group optionally having 1-2 hydrogen atoms replaced with C₁₋₆ alkyl,
R² is a chain aliphatic substituent having the —CH₂— fragment as the terminal member of the chain, to which chain a phenyl group is optionally attached optionally having 1-3 substituents selected from halogens and C₁₋₆ alkyl, and, if the chain contains at least 2 carbon atoms, in which chain there is optionally a bivalent link between the chain carbon atoms, said link selected from the oxygen atom —O—, sulfur atom —S— and C₃₋₆ cycloalkylene, wherein the R² substituent contains a total of 1-16 carbon atoms, and the hydrogen atom at one of the carbon atoms is replaced with an atom of ¹⁸F fluorine radioisotope,
R³ and R⁴ are combined to form a bivalent butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
and the hydrate or solvate thereof.

The disclosure also relates to a pharmaceutical composition containing the radioisotope-labelled compound of quaternary ammonium salt of a polycyclic aromatic amine according to the disclosure and a pharmaceutically acceptable carrier or diluent, in particular to a pharmaceutical composition in the form of a sterile solution. The sterile composition is provided in the final container (vial or syringe) placed in an external shielding.

Compounds according to the disclosure are manufactured by adapting general synthetic methods available in the field of organic chemistry. The method illustrated in scheme I uses a substituted compound with formula II, wherein R¹, R³ and R⁴ have the meanings indicated above for formula I, and wherein said compound with formula II is a derivative of acridine or phenanthridine, and it uses a compound GO^{A}—CH₂—R⁵(GO^{B}) with formula III, wherein the GO^{A} and GO^{B} symbols are leaving groups substitutable with a nucleophilic reagent in a nucleophilic substitution reaction. The R⁵—CH₂ fragment corresponds in terms of structure to the chain R² substituent defined above. The GO^{A} and GO^{B} leaving groups are structurally identical or different and are independently selected from alkylsulfonate leaving groups, fluoroalkylsulfonate leaving groups, arylsulfonate leaving groups, haloarylsulfonate leaving groups and halogen leaving groups with the exclusion of fluoride group. Preferably, leaving groups are methanesulfonate, ethanesulfonate, trifluoromethanesulfonate (triflate), pentafluoroethanesulfonate, toluenesulfonate (tosyl), 4-bromophenylsulfonate (brosyl), iodide and bromide groups.

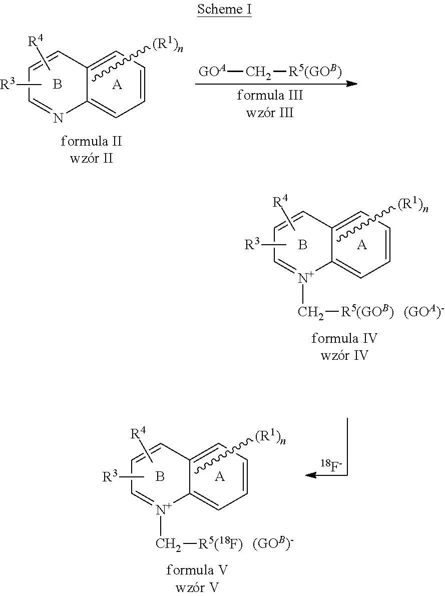

In the first step of the synthesis, the substitution of the GO^{A} group results in the formation of the acridinium or phenanthridinium compound with formula IV with the GO^{B} leaving group in the side chain, and in the second step of the synthesis, the compound with formula IV is reacted with the ¹⁸F⁻ fluoride anion to form the compound with formula V containing the ¹⁸F atom in the side chain attached to the quaternary nitrogen atom.

If required, R¹ groups in a compound with formula II, which could be engaged in side reactions with a compound with formula III, are routinely protected using known protective groups, for example those disclosed in the monograph “Protective Groups in Organic Synthesis” (Theodora W. Greene and Peter G. M. Wuts, 2nd edition, 1991, John Wiley & Sons, Inc.). The R¹ groups in the compound with formula II, that could be engaged in side reactions with the compound with formula III, are in particular primary and secondary amino groups, and optionally hydroxy groups.

Alternatively, the compounds according to the disclosure are manufactured by the method illustrated in scheme II using a substituted compound with formula II in which R¹, R³ and R⁴ are as defined above for formula I, and the GO^{C}—CH₂—R⁶ compound with formula VI, wherein R⁶ is a ¹⁸F fluorine atom or the CH₂-R⁶ fragment corresponds to the definition of R² in formula I, and the GO^{C} symbol is a leaving group substitutable by a nitrogen atom of the acridine or phenanthridine ring in a nucleophilic substitution reaction. The GO^{C} leaving group is selected from the leaving groups referred to above in the definition of the GO^{A} and GO^{B} groups. According to the method of scheme II, a compound with formula VII containing the ¹⁸F atom in the side chain is manufactured, wherein the R⁶ fragment is the ¹⁸F fluorine atom or the CH₂—R⁶ fragment corresponds to the definition of R² in formula I. Similarly to the reactions according to scheme I, if required, R¹ groups in the compound with formula II, that could be engaged in side reactions with the compound with formula VI, are protected following the standard procedure using the protection groups as above.

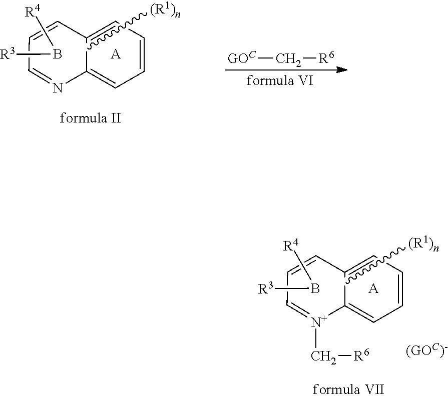

The compound according to the disclosure is obtained in the form of a quaternary ammonium salt together with a counterion, which is a mononegative anion of a stable organic or inorganic acid, corresponding to the leaving group departing in the last reaction step, in accordance with scheme I or II. The scope of the disclosure, however, is not limited to such salts, since the mononegative anion being the counterion in the quaternary salt can be replaced with another anion of a stable organic or inorganic acid as required using standard procedures. For example, the quaternary ammonium salt obtained by the method according to scheme I or II is dissolved in a solution of inorganic or organic salt containing the desired mononegative anion, or inorganic or organic salt containing the desired mononegative anion is added to the solution of the quaternary ammonium salt obtained by the method according to scheme I or II. Optionally, the quaternary ammonium salt obtained by the method according to scheme I or II shall be transformed into a salt containing the desired anion using ion exchangers, for example using the method disclosed in the paper entitled “An unusual substitution reaction directed by an intramolecular re-arrangement” (A. D. C. Parenty, L. V. Smith, L. Cronin), Tetrahedron, 61 (2005), pp. 8410-8418, using anion-exchange resin pre-treated with a solution of an alkali metal salt and acid of the desired anion, or recovered by way of elution with an appropriate buffer, in accordance with the procedure presented in the examples below.

Thus, the scope of subject disclosure covers a radioisotope-labelled compound of quaternary ammonium salt of a polycyclic aromatic amine with formula I, in which formula X⁻ is a mononegative anion of any stable organic or inorganic acid, preferably an anion selected from the group comprising an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid.

Said salts can form hydrates and/or solvates, which hydrates and/or solvates are also included in the scope of the disclosure.

Preferably, the disclosure relates to a radioisotope-labelled compound with formula I, in which formula I the wavy line indicates a single bond between the non-nodal carbon atom of a polycyclic aromatic system and the R¹ substituent,
R¹ is a substituent independently selected from hydrogen, halogens, C₁₋₄ alkoxy, a nitro group, an amino group optionally having 1 or 2 hydrogen atoms replaced with C₁₋₄ alkyl, a chain C₁₋₆ carbon group optionally having a halogen or C₁₋₄ alkanesulfonic substituent, and a phenyl group optionally having 1-3 substituents independently selected from halogens, a chain C₁₋₄ carbon substituent, a halogenated chain C₁₋₄ carbon substituent, an amino group optionally having 1-2 atoms of hydrogen replaced with C₁₋₄ alkyl,
R² is a chain aliphatic substituent having the —CH₂— fragment as the terminal member of the chain, to which chain a phenyl group is optionally attached optionally having 1-3 substituents selected from halogens and C₁₋₆ alkyl, and, if the chain contains at least 2 carbon atoms, in which chain there is optionally a bivalent link between the carbon atoms of the chain, said link selected from the oxygen atom —O— and sulfur atom —S—, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
and the hydrate or solvate thereof.

More preferably, the disclosure relates to a radioisotope-labelled compound with formula I, in which formula I the wavy line indicates a single bond between the non-nodal carbon atom of a polycyclic aromatic system and the R¹ substituent,
R¹ is a substituent independently selected from hydrogen, halogens, C₁₋₄ alkoxy, an amino group optionally having 1-2 hydrogen atoms replaced with C₁₋₄ alkyl, a chain C₁₋₄ carbon group optionally having a halogen substituent, and a phenyl group optionally having 1-3 substituents independently selected from halogens, a chain C₁₋₄ carbon substituent, a halogenated chain C₁₋₄ carbon substituent, C₁₋₄ alkoxy,
R² is a chain aliphatic substituent having the —CH₂— fragment as the terminal member of the chain, to which chain a phenyl group is optionally attached, optionally having 1-3 substituents selected from halogens and C₁₋₆ alkyl, and, if the chain contains at least 2 carbon atoms, in which chain there is optionally a bivalent link between carbon atoms of the chain, said link being the oxygen atom —O—, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
and the hydrate or solvate thereof.

Even more preferably, the compound of the disclosure is a radioisotope-labelled compound with formula I, in which formula I the wavy line indicates a single bond between the non-nodal carbon atom of a polycyclic aromatic system and the R¹ substituent,
R¹ is a substituent independently selected from hydrogen, halogens, C₁₋₄ alkoxy, an amino group optionally having 1-2 hydrogen atoms replaced with C₁₋₂ alkyl, a chain C₁₋₄ carbon group optionally having a halogen substituent, and a phenyl group optionally having 1-3 substituents independently selected from halogens, a chain C₁₋₄ carbon substituent and a halogenated chain C₁₋₄ carbon substituent,
R² is a chain aliphatic substituent having the —CH₂— fragment as the terminal member of the chain, to which chain a phenyl group is optionally attached optionally having 1-3 substituents selected from halogens and C₁₋₄ alkyl, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
and the hydrate or solvate thereof.

Particularly preferably, the compound of the disclosure is a radioisotope-labelled compound with formula I, in which formula I the wavy line indicates a single bond between the non-nodal carbon atom of a polycyclic aromatic system and the R¹ substituent,
R¹ is a substituent independently selected from hydrogen, halogens, an amino group optionally having 1-2 hydrogen atoms replaced with C₁₋₂ alkyl, a chain C₁₋₄ carbon group, and a phenyl group optionally having 1-3 substituents independently selected from a chain C₁₋₄ carbon substituent, a halogenated chain C₁₋₄ carbon substituent,
R² is a chain aliphatic substituent having the —CH₂— fragment as the terminal member of the chain, to which chain a phenyl group is optionally attached optionally having a halogen substituent, wherein the R² substituent contains a total of 1-16 carbon atoms, and the hydrogen atom at one of the carbon atoms is replaced with an atom of ¹⁸F fluorine radioisotope,
R³ and R⁴ are combined to form a butadienyl-1,3 substituent whose terminal carbon atoms are linked to the adjacent non-nodal carbon atoms of the B ring to form an aromatic C ring fused with the A and B ring system, having R¹ substituents at non-nodal carbon atoms,
n is an integer of 9,
X⁻ is a pharmaceutically acceptable counterion, which is an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid,
and the hydrate or solvate thereof.

The following examples, which include: (i) examples of the manufacturing of intermediates in the manufacturing method of radioisotope-labelled compounds according to the disclosure, (ii) examples of the manufacturing of standard compounds for radioisotope-labelled compounds according to the disclosure and (iii) examples of the manufacturing of radioisotope-labelled compounds according to the disclosure, and (iv) examples of the formulation of a pharmaceutical composition, and (v) examples of the use of said compounds in diagnostic techniques, illustrate in detail the solution according to the disclosure without limiting the scope thereof. Throughout the examples, DCM stands for dichloromethane and ACN for acetonitrile.

The NMR spectra as quoted, were recorded using the ‘zg30’ sequence for proton and fluorine spectra and the ‘zg_pi_CPD (Bruker 500 MHz) or ‘s2pul’ (Varian 300 MHz) sequence for carbon spectra, with hydrogen's proton decoupling. Proton spectra were calibrated against the TMS present in the sample (samples in chloroform) and against the residual signal of the DMSO solvent of 2.5 ppm (quintet). The external standard CFCl₃ was used for the fluorine spectra measurement.

### Example 1. 2-Fluoroethyl trifluoromethanesulfonate

Trifluoromethanesulfonic acid anhydride (5.000 g; 17.72 mmol) is added to a 100 ml round-bottom single-neck flask with a magnetic dipole and septum. The flask is cooled to a temperature of 0° C. using a cooling bath (crushed ice+NaCl aqueous solution). DCM is added using a needle and syringe. A solution of 2-fluoroethanol (1.050 g; 16.39 mmol) and pyridine (1.570 g; 19.85 mmol) in 6 ml DCM is added dropwise over 20 minutes, and the whole is stirred for 2 h at room temperature. At the end of the reaction, the mixture is poured into 20 ml of cold water for HPLC. The post-reaction mixture is transferred to a separation funnel and washed three times with water. The organic phase is dried over MgSO₄, and the solvent is then distilled off to dryness on a rotary evaporator to obtain 1.170 g of a pink liquid. Yield 36.4%

¹H NMR (CDCl₃, 500 MHz): δ 4.64-4.75 (m, 4H).

¹³C NMR (CDCl₃, 125 MHz): δ 74.7 (d, ²J_{C-F}=20.25 Hz), 79.9 (d, ¹J_{C-F}=173.88 Hz), 118.6 (q, ¹J_{C-F}=317.13).

¹⁹F NMR (CDCl₃, 470 MHz): —75.4 (s, 3F), −226.1-(−226.5) (m, 1F).

HR-MS C₃H₄F₄O₃S (196.12067 u).

### Example 2. 1,2-Bis(trifluoromethanesulfonyloxy)ethane

Trifluoromethanesulfonic acid anhydride (5.000 g; 17.72 mmol) is added to a 100 ml round-bottom single-neck flask with a magnetic dipole and septum. The reaction flask is cooled to a temperature of 0° C. using a cooling bath (crushed ice+NaCl aqueous solution). 12 ml DCM is added using a needle and syringe, after which a solution of ethane-1,2-diol (0.540 g; 8.70 mmol) and pyridine (1.400 g; 17.70 mmol) in 6 ml DCM is added dropwise over 20 minutes, and the whole is stirred for 1.5 h at room temperature. At the end of the reaction, the mixture is poured into 20 ml of cold water for HPLC. The post-reaction mixture is transferred to a separation funnel and washed three times with water. The organic phase is dried over MgSO₄, and the solvent is then distilled off to dryness on a rotary evaporator to obtain 2.200 g of a pink liquid. Yield 77.5%.

¹H NMR (CDCl₃, 500 MHz): δ 4.77 (s, 4H).

¹³C NMR (CDCl₃, 125 MHz): δ 71.87, 118.5 (q, ¹J_{C-F}=317.38 Hz).

¹⁹F NMR (CDCl₃, 470 MHz): —74.7 (s, 6F).

### Example 3. 1,6-Bis(trifluoromethanesulfonyloxy)hexane

Trifluoromethanesulfonic acid anhydride (7.000 g; 24.81 mmol) is added to a 100 ml round-bottom single-neck flask with a magnetic dipole and septum. The reaction flask is cooled to a temperature of 0° C. using a cooling bath (crushed ice+NaCl aqueous solution). 12 ml DCM is added using a needle and syringe, after which a solution of hexane-1,6-diol (1.400 g; 11.85 mmol) and pyridine (1.450 g; 18.33 mmol) in 50 ml DCM is added dropwise over 20 minutes. The whole is stirred for 3 h at room temperature. At the end of the reaction, the mixture is poured into 20 ml of cold water for HPLC. The post-reaction mixture is transferred to a separation funnel and washed three times with water. The organic phase is dried over MgSO₄, and the solvent is then distilled off to dryness on a rotary evaporator to obtain 2.000 g of the product. Yield 44.2%.

¹H NMR (CDCl₃, 500 MHz): δ 1.51 (quint, 4H, ³J_{H-H}=4 Hz), 1.80-1.90 (m, 4H), 4.55 (t, ³J_{H-H}=6.5 Hz, 4H).

¹³C NMR (CDCl₃, 125 MHz): δ 24.5, 28.9, 77.2, 118.6 (q, ¹J_{C-F}=317.25 Hz).

¹⁹F NMR (CDCl₃, 282 MHz): —75.0.

HR-MS C₈H₁₂F₆O₆S₂ (382.29770 u).

### Example 4. 1,3-Bis(trifluoromethanesulfonyloxy)propane

Trifluoromethanesulfonic acid anhydride (2.104 g; 7.46 mmol) is added to a 100 ml round-bottom single-neck flask with a magnetic dipole and septum. The reaction flask is cooled to a temperature of 0° C. using a cooling bath (crushed ice+NaCl aqueous solution). 12 ml DCM is added using a needle and syringe, after which a solution of propane-1,3-diol (0.261 g; 3.43 mmol) and pyridine (0.620 g; 7.84 mmol) in 6 ml DCM is added dropwise over 20 minutes, and the whole is stirred for 1.5 h at room temperature. At the end of the reaction, the mixture is poured into 20 ml of cold water for HPLC, transferred to a separation funnel and washed three times with water. The organic phase is dried over MgSO₄, and the solvent is then distilled off to dryness on a rotary evaporator to obtain 0.938 g of the title compound in the form of a pink liquid. Yield 80.38%.

¹H NMR (CDCl₃, 500 MHz): δ 2.37 (q, ³J_{H-H}=5.5 Hz, 2H), 4.68 (t, ³J_{H-H}=6.0 Hz, 4H).

¹³C NMR (CDCl₃, 125 MHz): δ 29.3, 71.5, 118.6 (q, ¹J_{C-F}=319.60 Hz).

¹⁹F NMR (CDCl₃, 470 MHz): —74.6 (s, 6F).

### Example 5. 1,16-Bis(trifluoromethanesulfonyloxy)hexadecane

Trifluoromethanesulfonic acid anhydride (2.50 g; 8.86 mmol) and hexadecane-1,16-diol (0.252 g; 0.98 mmol) are added to a 100 ml round-bottom single-neck flask with a magnetic dipole and septum. The reaction is carried out at room temperature for 4 h. At the end of the reaction, the mixture is poured into 20 ml DCM and cold water for HPLC. The post-reaction mixture is transferred to a separation funnel and washed three times with water. The phase is dried over MgSO₄, and the solvent is then distilled off to dryness on a rotary evaporator to obtain 0.301 g of the product. Yield 17.4%.

¹H NMR (CDCl₃, 500 MHz): δ 1.20-1.30 m (20H), 1.41 (quint, 4H, ³J_{H-H}=4 Hz), 1.82 (quint, ³J_{H-H}=4 Hz, 4H), 4.54 (t, ³J_{H-H}=6.5 Hz, 4H).

¹³C NMR (CDCl₃, 125 MHz): δ 25.0, 28.8, 29.2, 29.3, 29.4, 29.6, 29.6, 77.8, 118.6 (q, ¹J_{C-F}=317.25 Hz).

¹⁹F NMR (CDCl₃, 470 MHz): —74.9.

### Example 6. 2-(4-Fluorophenyl)ethyl trifluoromethanesulfonate

Trifluoromethanesulfonic acid anhydride (2.000 g; 7.09 mmol) is added to a 50 ml round-bottom single-neck flask with a magnetic dipole and septum. The solution is cooled to a temperature of 0° C. using a cooling bath (ice+NaCl), 2-(4-fluorophenyl)ethanol (0.921 g; 6.57 mmol) is added dropwise to the cooled solution over about 10 minutes, and the contents of the flask are stirred for 4 h at room temperature. At the end of the reaction, the mixture is poured into 20 ml DCM and cold water for HPLC. The reaction mixture is transferred to a separation funnel and washed repeatedly with water to remove the acid. The phase is dried over MgSO₄, and the solvent is then distilled off on a rotary evaporator to obtain 0.999 g of the product. Yield 55.8%.

¹H NMR (CDCl₃, 500 MHz): δ 3.10 (t, ³J_{H-H}=7.0 Hz), 4.66 (td, ³J_{H-H}=7.0 Hz, ⁴J_{H-F}=0.5 Hz, 2H), 7.03 (dd, ³J_{H-F}=9.0 Hz, ⁴J_{H-H}=5.0 Hz, 2H), 7.16-7.20 (m, ³J_{H-H}=7.0 Hz, 2H).

¹³C NMR (CDCl₃, 125 MHz): δ 35.24, 77.55, 116.2 (d, ²J_{C-F}=21.5 Hz), 118.6 (q, ¹J_{C-F}=317.13), 130.8090 (d, ³J_{C-F}=8.0 Hz), 130.8095 (d, ⁴J_{C-F}=3.4 Hz), 162.5 (d, ¹J_{C-F}=246.2 Hz).

¹⁹F NMR (CDCl₃, 282 MHz): —74.8 (s, 3F), −115.0 (tt, ³J_{F-H}=8.8 Hz, ⁴J_{F-H}=5.0 Hz, 1F).

### Example 7. 5-(2-Fluoroethyl)phenanthridinium p-toluenesulfonate

Phenanthridine (0.410 g; 2.29 mmol) is added to a 100 ml round-bottom single-neck flask in an inert gas atmosphere, after which 1 ml of toluene is added three times using a needle and syringe, which is then distilled off to dryness. Phenanthridine dissolved in DMF is added dropwise to 2-fluoroethyl tosylate (1.000 g; 4.58 mmol) in DMF, contained in a 100 ml round-bottom single-neck flask equipped with a magnetic dipole and mounted on a magnetic stirrer. The reaction is carried out in an inert gas atmosphere for 168 h at 105° C. At the end of the reaction, the yellow to brown solution is concentrated on a rotary evaporator to remove DMF. The residue is cooled to room temperature and dissolved in cold acetone (about 4 ml). Diethyl ether (about 60 ml) is added, and this is cooled in a refrigerator. The precipitated product is filtered off. 0.20 g of the title compound is obtained (22.0% yield).

¹H NMR ((CD₃)₂SO, 500 MHz): δ 5.09 (td, ²J_{H-F}=47 Hz, ³J_{H-H}=4.5 Hz, 2H), 5.53 (td, ³J_{H-F}=26 Hz, ³J_{H-H}=4.5 Hz, 2H), 7.94-8.04 (m, 4H), 8.10-8.17 (m, 4H), 8.20-8.30 (m, 4H), 8.41-8.45 (m, 1H), 8.54 (dd, ³J_{H-H}=8 Hz, ⁴J_{H-H}=0.5 Hz, 1H), 8.62-8.69 (m, 2H), 9.00-9.06 (m, 2H), 9.16 (d, ³J_{H-H} 8 Hz, 1H), 9.21 (dd, ³J_{H-H}=8 Hz, ⁴J_{H-H}=2 Hz), 10.37 (s, 1H).

¹⁹F NMR ((CD₃)₂SO, 470 MHz): δ −221.1 (tt, ²J_{F-H}=47.1 Hz, ³J_{H-H}=25.9 Hz, 1F).

HR-MS C₇H₇S₁O₃⁻ (171.01104 u), found: 171.01122 u, C₁₅H₁₃N₁F₁⁺ (226.10265 u), found: 226.10250 u.

### Example 8. 6-Phenyl-5-(2-fluoroethyl)phenanthridinium trifluoromethanesulfonate

2-Fluoroethyl trifluoromethanesulfonate (0.290 g; 1.48 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate). A solution of 6-phenylphenanthridine (0.210 g; 0.82 mmol) in 6 ml DCM is added dropwise to the cooled solution over about 20 minutes. The contents of the flask are stirred for 216 h. At the end of the reaction, cold Et₂O is added to the reaction mixture. The mixture is left in the refrigerator for 30 min, after which the product is filtered under reduced pressure. 0.200 g of the title compound is obtained (53.9% yield).

¹H NMR ((CD₃)₂SO, 500 MHz): δ 4.95 (dt, ²J_{H-F}=46.5 Hz, ³J_{H-H}=5.0 Hz, 2H), 5.30 (dbs, ³J_{H-F}=23.5 Hz, 2H), 7.56 (d, ³J_{H-H}=8 Hz, 1H), 7.75-7.88 (m, 5H), 7.96 (t, ³J_{H-H}=7.5 Hz, 1H), 8.15-8.23 (m, 2H), 8.48 (ddd, ³J_{H-H}=8.5 Hz, ³J_{H-H}=7.0 Hz, ⁴J_{H-H}=1.5 Hz, 1H), 8.75 (d, ³J_{H-H}=9 Hz, 1H), 9.28 (d, ³J_{H-H}=8 Hz, 1H), 9.32 (dd, ³J_{H-H}=8.5 Hz, ⁴J_{H-H}=1.5 Hz, 1H).

¹³C NMR ((CD₃)₂SO, 125 MHz): δ 54.1 (d, ²J_{C-F}=21.00 Hz), 81.3 (d, ¹J_{C-F}=170.00 Hz), 121.1, 123.3, 124.8, 125.5, 126.0, 128.9, 129.2, 130.4, 130.6, 130.9, 131.4, 132.3, 132.9, 134.2, 134.7, 137.8, 165.3.

¹⁹F NMR ((CD₃)₂SO, 470 MHz): δ −78.1 (s, 3F), −220.4-−220.8 (tt, ²J_{F-H}=49.0 Hz, ³J_{H-H}=22.1 Hz, 1F).

HR-MS C₁O₃F₃S₁⁻ (148.95148 u), found: 148.95106 u, C₂₁H₁₇FN⁺ (302.36423 u).

### Example 9. 10-(2-Fluoroethyl)acridinium trifluoromethanesulfonate

2-Fluoroethyl trifluoromethanesulfonate (0.270 g; 1.38 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate). A solution of acridine (0.220 g; 1.23 mmol) in 6 ml DCM is added dropwise to the cooled solution over about 20 minutes, after which the contents of the flask are stirred for 72 h. At the end of the reaction, 20 ml of cold Et₂O is added to the reaction mixture, the mixture is left in the refrigerator for 30 min, and then the product is filtered off under reduced pressure. 0.200 g of the title compound is obtained. Yield 43.4%.

¹H NMR ((CD₃)₂SO, 500 MHz): δ 5.15 (dt, ²J_{H-F}=47 Hz, ³J_{H-H}=4.5 Hz, 2H), 5.91 (dt, ³J_{H-F}=25.5 Hz, J=4.5 Hz, 2H), 8.06 (dd, ³J_{H-H}=8.5 Hz, ³J_{H-H}=7.0 Hz, 2H), 8.48 (ddd, ³J_{H-H}=9.5 Hz, ³J_{H-H}=7.0 Hz, ⁴J_{H-H}=1.5 Hz, 2H), 8.67 (dd, ³J_{H-H}=8 Hz, ⁴J_{H-H}=1.5 Hz, 2H), 8.80 (d, ³J_{H-H}=9.5 Hz, 2H), 10.26 (s, 1H).

¹³C NMR ((CD₃)₂SO, 125 MHz): δ 49.9 (d, ²J_{C-F}=19.12 Hz), 82.3 (d, ¹J_{C-F}=168.88 Hz), 118.9, 126.4, 127.8, 132.0, 139.4, 141.5, 152.1.

¹⁹F NMR ((CD₃)₂SO, 470 MHz): δ −78.1 (s, 3F), −221.5 (tt, ²J_{F-H}=47 Hz, ²J_{F-H}=25.5 Hz, 1F).

HR-MS C₁O₃F₃S₁⁻ (148.95148 u), C₁₅H₁₃N₁F₁⁺ (226.10265 u), found: 226.10250 u.

### Example 10. 3,6-Bis(dimethylamino)-10-(2-fluoroethyl)acridinium trifluoromethanesulfonate

2-Fluoroethyl trifluoromethanesulfonate (0.218 g; 1.11 mmol) is added to a 50 ml round-bottom single-neck flask with a magnetic dipole. 10 ml DCM is added using a needle and syringe. The solution is cooled to a temperature of −56° C. using a cooling bath (liquid nitrogen+ethyl acetate). A solution of Acridinium Orange (0.137 g; 0.52 mmol) in 10 ml DCM is added dropwise to the cooled solution over about 20 minutes, after which the contents of the flask are stirred for 72 h. At the end of the reaction, 20 ml of cold Et₂O is added to the reaction mixture, the mixture is left in the refrigerator for 30 min, and then the product is filtered off under reduced pressure. 0.104 g of the title compound is obtained (57.5% yield).

¹H NMR ((CD₃)₂SO, 500 MHz): δ 3.19 (s, 12H), 4.90-5.10 (m, 4H), 6.54 (s, 2H), 7.10 (d, ³J_{H-H}=9 Hz, 2H), 7.73 (d, ³J_{H-H}=9 Hz, 2H), 8.56 (s, 1H).

¹³C NMR ((CD₃)₂SO, 125 MHz): δ 40.2, 46.8 (d, ²J_{C-F}=19.9 Hz), 81.6 (d, ¹J_{C-F}=167.6 Hz), 92.9, 114.0, 116.2, 120.7 (q, ¹J_{C-F}=320 Hz), 132.8, 142.6, 143.0, 155.2.

¹⁹F NMR ((CD₃)₂SO, 282 MHz): δ −77.71 (s, 3F), −221.3 (tt, ²J_{F-H}=49.4 Hz, ³J_{H-H}=25.1 Hz, 1F).

HR-MS C₃F₃S₁O₃⁻ (148.95148 u), found: 148.95106 u, C₁₉H₂₃FN₃ (312.40387 u).

### Example 11. 5-(2-Trifluoromethylsulfonyloxyethyl)phenanthridinium trifluoromethanesulfonate

1,2-Bis(trifluoromethanesulfonyloxy)ethane (0.310 g, 0.95 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate). A solution of phenanthridine (0.170 g, 0.95 mmol) in 10 ml DCM is added dropwise to the cooled solution over about 20 minutes, after which the contents of the flask are stirred for 48 h. At the end of the reaction, 20 ml of cold Et₂O is added to the reaction mixture, the mixture is left in the refrigerator for 30 min, and then the product is filtered off under reduced pressure. 0.160 g of the title compound is obtained (33.3% yield).

¹H NMR ((CD₃)₂SO, 500 MHz): δ 4.88 (t, ³J_{H-H}=4.5 Hz, 2H), 5.48 (t, ³J_{H-H}=4.5 Hz, 2H), 8.10-8.25 (m, 3H), 8.46 (ddd, ³J_{H-H}=8.0 Hz, ³J_{H-H}=7.0 Hz, ⁴J_{H-H}=1.0 Hz, 2H), 8.66 (dd, ³J_{H-H}=8.0 Hz, ⁴J_{H-H}=1.0 Hz, 2H), 9.19 (d, ³J_{H-H}=8.5 Hz, 1H), 9.24 (dd, ³J_{H-H}=8.5 Hz, ⁴J_{H-H}=2.0 Hz, 1H), 10.23 (s, 1H).

¹³C NMR ((CD₃)₂SO, 125 MHz); δ 56.9, 73.1, 120.0, 120.7 (q, ¹J_{C-F}=320.25), 123.2, 123.5, 125.1, 126.0, 130.4, 130.5, 132.0, 133.1, 133.6, 134.8, 138.6, 156.6.

¹⁹F NMR ((CD₃)₂SO, 470 MHz); δ −78.1 (s, 3F).

HR-MS C₁F₃S₁O₃⁻ (148.95148 u), found: 148.95147 u, C₁₆H₁₃N₁S₁O₃F₃⁺ (356.05628 u), found: 356.05616 u.

### Example 12. 6-Phenyl-5-(2-trifluoromethylsulfonyloxyethyl)phenanthridinium trifluoromethanesulfonate

1,2-Bis(trifluoromethanesulfonyloxy)ethane (0.404 g; 1.24 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate), 6-phenylphenanthridine solution (0.170 g; 0.67 mmol) in 6 ml DCM is added dropwise over about 20 minutes to the cooled solution, and the contents of the flask are then stirred for 96 h. At the end of the reaction, the mixture is concentrated to 8 ml, 40 ml of cold Et₂O is added, and this is left in the refrigerator for 30 minutes. The product is filtered under reduced pressure to obtain 0.200 g of the title compound (51.7% yield).

¹H NMR ((CD₃)₂SO, 500 MHz): δ 4.70 (t, 2H, ³J_{H-H}=4.5 Hz), 5.26 (bs, 2H), 7.58 (d, 1H, ³J_{H-H}=8.5 Hz), 7.72-7.88 (m, 5H), 7.98 (t. 1H, J=7.5 Hz), 8.15-8.25 (m, 2H), 8.42 (t, 1H, J=8.0 Hz), 8.65 (d, 1H, ³J_{H-H}=9.5 Hz), 9.29 (d, 1H, ³J_{H-H}=8.5 Hz), 9.34 (d, 1H, ³J_{H-H}=9.5 Hz).

¹⁹F NMR ((CD₃)₂SO, 470 MHz): δ −78.1 (s, 3F).

MS-HR C₁O₃F₃S₁⁻ (148.95148), found: 148.95106, C₂₂H₁₇F₃NO₃S (432.43484 u) found: 432.08751 u.

### Example 13. 10-(2-Trifluoromethylesulfonyloxyethyl)acridinium trifluoromethanesulfonate

1,2-Bis(trifluoromethanesulfonyloxy)ethane (0.29 g, 0.89 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate), acridine solution (0.120 g; 0.67 mmol) in 6 ml DCM is added dropwise over about 20 minutes to the cooled solution, and the contents of the flask are then stirred for 48 h. At the end of the reaction, the mixture is concentrated to 4 ml, 20 ml of cold Et₂O is added, and this is left in the refrigerator for 30 minutes. The product is filtered under reduced pressure to obtain 0.03 g of the title compound. Yield 8.86%.

¹H NMR ((CD₃)₂SO, 500 MHz): δ 5.39 (t, ³J_{H-H}=5.0 Hz, 2H), 6.02 (t, ³J_{H-H}=5.0 Hz, 2H), 8.00-8.10 (dd, ³J_{H-H}=8.0 Hz, ³J_{H-H}=7.0 Hz, 2H), 8.15 (ddd, ³J_{H-H}=9.0 Hz, ³J_{H-H}=7.0 Hz, ⁴J_{H-H}=1.5 Hz, 2H), 8.32 (d, ³J_{H-H}=9.5 Hz, 2H), 8.47 (dd, ³J_{H-H}=8.5 Hz, ⁴J_{H-H}=1 Hz, 2H), 9.99 (s, 1H).

¹⁹F FMR ((CD₃)₂SO, 470 MHz): δ −78.1 (s, 3F).

MS-HR C₃F₃S₁O₃⁻ (148.95148), found: 148.95106, C₁₆H₁₃F₃NO₃S⁺ (356.338981 u).

### Example 14. 3,6-Bisdimethylamino-10-(2-trifluoromethylesulfonyloxyethyl)acridinium trifluoromethanesulfonate

1,2-Bis(trifluoromethanesulfonyloxy)ethane (0.355 g, 1.09 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate), and a solution of Acridinium Orange (0.207 g, 0.78 mmol) in 6 ml DCM is added dropwise to the cooled solution over about 20 minutes. The contents of the flask are stirred for 96 h and, at the end of the reaction, concentrated to 4 ml, and 20 ml of cold Et₂O is added. The mixture is left in the refrigerator for 30 min, after which the product is filtered under reduced pressure to obtain 0.310 g of the title compound. Yield 67.2%.

¹H NMR ((CD₃)₂SO, 500 MHz): δ 3.27 (s, 12H), 4.85 (t, ³J_{H-H}=4.5 Hz, 2H), 5.15 (bs, 2H), 6.63 (s, 2H), 7.25 (d, ³J_{H-H}=9.0 Hz, 2H), 7.89 (d, ³J_{H-H}=7.5 Hz, 2H), 8.76 (s, 1H).

¹³C NMR ((CD₃)₂SO, 125 MHz): δ 40.4, 57.8, 73.2, 93.1, 114.2, 116.4, 120.7 (q, ¹J_{C-F}=320 Hz), 133.0, 143.0, 155.5.

¹⁹F NMR ((CD₃)₂SO, 470 MHz): δ −78.1 (s, 3F).

### Example 15. 5-(3-Fluoropropropyl)phenanthridinium trifluoromethanesulfonate

The title compound is obtained by following the procedure described in example 7, but using 3-fluoropropropyl trifluoromethanesulfonate (0.229 g, 1.09 mmol) instead of 2-fluoroethyl trifluoromethanesulfonate and phenanthridine (0.230 g, 1.28 mmol). Yield 71.0%.

¹H NMR ((CD₃)₂SO, 500 MHz): δ 2.46-2.58 (m, 2H), δ 4.73 (dt, ²J_{H-F}=47 Hz, ³J_{H-H}=5.5 Hz, 2H), 5.24 (t, ³J_{H-H}=7 Hz, 2H), 8.09-8.14 (m, 2H), 8.14-8.19 (m, 1H), 8.37-8.43 (m, 1H), 8.59 (dd, ³J_{H-H}=8 Hz, ⁴J_{H-H}=1 Hz, 1H), 8.63 (d, ³J_{H-H}=8 Hz, 1H), 9.12 (d, ³J_{H-H}=8 Hz, 1H), 9.18 (dd, ³J_{H-H}=8 Hz, ⁴J_{H-H}=1 Hz, 1H), 10.37 (s, 1H).

¹³C NMR ((CD₃)₂SO, 125 MHz): δ 29.8 (d, ¹J_{C-F}=19.375 Hz), δ 55.0 (d, ¹J_{C-F}=4.5 Hz), 81.5 (d, ¹J_{C-F}=161.0 Hz), 119.8, 120.7 (q, ¹J_{C-F}=320.25 Hz), 123.1, 123.7, 125.1, 125.9, 130.3, 130.4, 132.1, 132.8, 133.1, 134.4, 138.1, 155.9.

¹⁹F NMR ((CD₃)₂SO, 470 MHz): δ −219.7-−220.1 (m, 1F), −77.72 (s, 3F).

### Example 16. 5-[2-(4-Fluorophenyl)ethyl]phenanthridinium trifluoromethanesulfonate

The title compound is obtained by following the procedure described in example 7, but using 2-(4-fluorophenyl)ethyl trifluoromethanesulfonate (0.174 g, 0.64 mmol) instead of 2-fluoroethyl trifluoromethanesulfonate and phenanthridine (0.147 g, 0.82 mmol). 0.198 g of the product is obtained (53.7% yield).

¹H NMR ((CD₃)₂SO, 500 MHz): δ 3.41 (³J_{H-H}=7.5 Hz. 2H). 5.33 (t. ³J_{H-H}=7.5 Hz. 2H), 7.09-7.14 (m. 2H). 7.27-7.33 (m, 2H), 8.10 (ddd, ³J_{H-H}=8.0 Hz, ³J_{H-H}=7.5 Hz, ⁴J_{H-H}=1.0 Hz, 1H), 8.14 (ddd, ³J_{H-H}=8.0 Hz, ³J_{H-H}=7.5 Hz, ⁴J_{H-H}=1.5 Hz, 1H), 8.19 (ddd, ³J_{H-H}=9.0 Hz, ³J_{H-H}=7.5 Hz, ⁴J_{H-H}=1.5 Hz, 1H), 8.41 (ddd, ³J_{H-H}=8.5 Hz, ³J_{H-H}=7.0 Hz, ⁴J_{H-H}=1.5 Hz, 1H), 8.48 (dd, ³J_{H-H}=8.0 Hz, ⁴J_{H-H}=1.0 Hz, 1H), 8.74 (dd, ³J_{H-H}=8.5 Hz, ⁴J_{H-H}=1.0 Hz, 1H), 9.15 (d, ³J_{H-H}=8.5 Hz, 1H), 9.21 (dd, ³J_{H-H}=8 Hz, ⁴J_{H-H}=1.5 Hz, 1H), 10.13 (s, 1H).

¹³C NMR ((CD₃)₂SO, 125 MHz): δ 34.1, 58.5, 115.4 (d, ²J_{C-F}=21.3 Hz), 120.1, 120.7 (q, ¹J_{C-F}=320.25 Hz), 123.2, 123.4, 125.1, 125.8, 130.5, 130.4, 131.0 (d, ³J_{C-F}=8.2 Hz), 132.2, 132.6 (d, ⁴J_{C-F}=3.1 Hz), 132.2 132.7, 132.9, 134.4, 138.2, 155.5, 161.3 (d, ¹J_{C-F}=243.2 Hz).

¹⁹F NMR ((CD₃)₂SO, 470 MHz): δ −77.72 (s, 3F), −115.6-−115.5 (m, 1F).

### Example 17. 10-(2-Iodoethyl)acridinium iodide

1.2-Diiodoethane (1.033 g, 3.66 mmol) is added to a 50 ml round-bottom single-neck flask with a magnetic dipole. Acridine (0.406 g, 2.26 mmol) dissolved in 10 ml toluene is added using a needle and syringe, and the reaction is carried out at solvent boiling point for 10 h. The mixture is cooled, the precipitate formed is filtered off and washed with diethyl ether. 0.433 g of a compound with 33% purity (according to HPLC) is obtained. 1H NMR ((CD₃)₂SO, 500 MHz): δ 3.79 (t, ³J_{H-H}=7.5 Hz, 2H), 5.76 (t, ³J_{H-H}=7.5 Hz, 2H), 8.03 (dd, ³J_{H-H}=8.0 Hz, ³J_{H-H}=7.0 Hz, 2H), 8.48 (ddd, ³J_{H-H}=8.5 Hz, ³J_{H-H}=7.5 Hz, ⁴J_{H-H}=1.5 Hz, 2H), 8.63 (dd, ³J_{H-H}=8.5 Hz, ⁴J_{H-H}=1.5 Hz, 2H), 8.69 (d, ³J_{H-H}=9.5 Hz, 2H) 10.20 (s, 1H).

### Example 18. 5-(16-Trifluoromethylsulfonyloxyhexadecyl)phenanthridinium trifluoromethanesulfonate

1,16-Bis(trifluoromethanesulfonyloxy)hexadecane (0.145 g, 0.28 mmol) is added to a 50 ml round-bottom single-neck flask with a magnetic dipole. 15 ml DCM is added using a needle and syringe. A solution of phenanthridinium (0.033 g, 0.18 mmol) in 15 ml DCM is added dropwise to the solution over about 20 minutes, after which the contents of the flask are stirred for 96 h. At the end of the reaction, DCM is evaporated, and the residue is washed with diethyl ether. 0.038 g of the title compound is obtained. Yield 19.5%.

¹H NMR ((CD₃)₂SO, 500 MHz): δ 1.1-1.4 (m, 20H), 1.45 (bs, 2H), 2.06 (bs, 2H), 4.5-4.7 (bs, 2H), 5.08 (t, 2H, ³J_{H-H}=7.5 Hz), 8.07-8.17 (m, 3H), 8.40 (ddd ³J_{H-H}=8.5 Hz, ³J_{H-H}=7.5 Hz, ³J_{H-H}=1.5 Hz, 1H), 8.58 (d, ³J_{H-H}=8.0 Hz, 1H), 8.64 (d, ³J_{H-H}=8.5 Hz, 1H), 9.14 (d, ³J_{H-H}=8.0 Hz, 1H), 9.19 (dd, ³J_{H-H}=8.0 Hz, ⁴J_{H-H}=1.5 Hz, 1H), 10.32 (s, 1H).

¹³C NMR ((CD₃)₂SO, 125 MHz): δ 24.7, 25.5, 25.7, 25.9, 28.5, 28.6, 28.8, 28.86, 28.91, 28.96, 29.00, 29.02, 29.06, 29.09, 60.7, 76.2, 119.4, 120.7 (q, ¹J_{C-F}=320.25), 123.2, 123.7, 125.0, 125.9, 130.3, 130.4, 132.1, 132.8, 133.1, 134.4, 138.0, 155.3.

### Example 19. 6-[4-(Chloromethyl)phenyl]-5-(2-fluoroethyl)phenanthridinium trifluoromethanesulfonate

2-Fluoroethyl trifluoromethanesulfonate (0.091 g, 0.46 mmol) is added to a 50 ml round-bottom single-neck flask with a magnetic dipole. 6 ml DCM is added using a needle and syringe. A solution of 6-[4-(chloromethyl)phenyl]phenanthridine (0.071 g, 0.23 mmol) in 6 ml DCM is added dropwise to the cooled solution over about 20 minutes. The contents of the flask are mixed for 70.5 h. At the end of the reaction, cold Et₂O (40 ml) is added to the reaction mixture. The mixture is left in the refrigerator for 30 min, after which the product is filtered under reduced pressure. 0.019 g of the title compound is obtained (15.6% yield).

¹H NMR ((CD₃)₂SO, 500 MHz): δ 4.93 (dt, ²J_{H-F}=47.0 Hz, ³J_{H-H}=4.5 Hz, 2H), 4.99 (s, 2H), 5.27 (bd, ³J_{H-F}=23.5 Hz), 7.54 (dd, ³J_{H-H}=8.5 Hz, ⁴J_{H-H}=1.0 Hz, 1H), 7.80 (d, ³J_{H-H}=8.0 Hz, 2H), 7.87 (d, ³J_{H-H}=8.0 Hz, 2H), 7.97 (ddd, ³J_{H-H}=8.5 Hz, ³J_{H-H}=7.0 Hz, ⁴J_{H-H}=1.0 Hz, 1H), 8.17-8.23 (m, 2H), 8.41 (ddd, ³J_{H-H}=8.0 Hz, ³J_{H-H}=7.0 Hz, ⁴J_{H-H}=1.5 Hz, 1H), 8.74 (d, ³J_{H-H}=9.5 Hz, 1H), 9.28 (d, ³J_{H-H}=8.5 Hz, 1H), 9.33 (dd, ³J_{H-H}=7.0 Hz, ⁴J_{H-H}=2.0 Hz, 1H).

¹³C NMR ((CD₃)₂SO, 125 MHz): δ 45.3, 54.2 (d, ²J_{C-F}=21.3 Hz), 81.3 (d, ²J_{C-F}=170.7 Hz), 121.1, 120.7 (q, ¹J_{C-F}=320.25 Hz), 120.8, 123.3, 124.9, 125.4 126.0, 129.4, 130.5, 130.6, 130.7, 132.3, 132.8, 134.1, 134.7, 137.8, 140.9, 164.9.

¹⁹F NMR ((CD₃)₂SO, 282 MHz): δ −77.7 (s, 3F), −220.2 (tt, 46.9, 24.0, 1F).

HR-MS C₁O₃F₃S₁⁻ (148.95148), found: 148.95106, C₂₂H₁₈FClN⁺ (350.11063), found: 350.11040.

### Example 20. 9-Chloro-10-[2-(4-fluorophenyl)ethyl]acridinium trifluoromethanesulfonate

2-(4-Fluorophenyl)ethyl trifluoromethanesulfonate (0.068 g, 0.25 mmol) is added to a 50 ml round-bottom single-neck flask with a magnetic dipole. 6 ml DCM is added using a needle and syringe. A solution of 9-chloroacridine (0.057 g, 0.27 mmol) in 6 ml DCM is added dropwise to the solution over about 20 minutes. The contents of the flask are mixed for 19 h. At the end of the reaction, cold Et₂O (40 ml) is added to the reaction mixture. The mixture is left in the refrigerator for 30 min, after which the product is filtered under reduced pressure. 0.001 g of the title compound is obtained (0.6% yield).

HR-MS C₁O₃F₃S₁⁻ (148.95148), found: 148.95106, C₂₁H₁₆FClN⁺ (336.09498), found: 336.09481.

### Example 21. 6-(4-Butylphenyl)-5-[2-(4-fluorophenyl)ethyl]phenanthridinium trifluoromethanesulfonate

The title compound is obtained by following the procedure described in example 20, but using 6-(4-butylphenyl)phenanthridine (0.103 g, 0.33 mmol) instead of 9-chloroacridine and 2-(4-fluorophenyl)ethyl trifluoromethanesulfonate (0.1955 g, 0.72 mmol). 0.032 g of the product is obtained (16.5% yield).

¹H NMR ((CD₃)₂SO, 500 MHz): δ 0.98 (t, ³J_{H-H}=7.5 Hz, 3H), 1.39 (sext, ³J_{H-H}=7.5 Hz, 2H), 1.71 (quint, ³J_{H-H}=7.5 Hz, 2H), 2.82 (t, ³J_{H-H}=7.5 Hz, 3H), 3.26 (t, ³J_{H-H}=7.5 Hz, 2H), 4.94 (bs, 2H), 6.97-7.10 (m, 4H), 7.60-7.75 (m, 5H), 7.97 (t, ³J_{H-H}=8.0 Hz, 1H), 8.20-8.30 (m, 2H), 8.40 (td, ³J_{H-H}=8.0 Hz, ⁴J_{H-H}=1.0 Hz, 1H), 8.90 (d, ³J_{H-H}=8.5 Hz, 1H), 9.26 (d, ³J_{H-H}=10.00 Hz, 1H), 9.33 (dd, ³J_{H-H}=8.5 Hz, ⁴J_{H-H}=1.0 Hz, 1H).

¹³C NMR ((CD₃)₂SO, 125 MHz): δ 13.8, 21.6, 33.0, 33.5, 34.7, 55.3, 115.3 (d, ²J_{C-F}=21.3, 21.00 Hz), 121.0, 120.7 (q, ¹J_{C-F}=320.25 Hz), 123.2, 124.9, 125.6 125.9, 128.4, 129.2, 133.0, 133.5, 130.5 (d, ³J_{C-F}=8.2 Hz), 132.5, 132.7, 132.7 (d, ⁴J_{C-F}=2.9 Hz), 133.8, 134.5, 137.5, 161.3 (d, ¹J_{C-F}=243.5 Hz), 164.6.

¹⁹F NMR ((CD₃)₂SO, 470 MHz); δ −77.7 (s, 3F), −115.5-−115.4 (m, 1F).

### Example 22. 6-Phenyl-5-(3-fluoropropyl)phenanthridinium trifluoromethanesulfonate

3-Fluoropropropyl trifluoromethanesulfonate (0.159 g, 0.76 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate). A solution of 6-phenylphenanthridine (0.149 g, 0.58 mmol) in 6 ml DCM is added dropwise to the cooled solution over about 20 minutes. The contents of the flask are mixed for 96 h. At the end of the reaction, the product is concentrated on an evaporator, cold Et₂O is added, and the mixture is left in the refrigerator for 30 minutes. The precipitated product is filtered off under reduced pressure. 0.136 g of the title compound is obtained (50.0% yield).

¹H NMR ((CD₃)₂SO, 500 MHz): δ 2.36 (dq, ³J_{H-F}=27.0 Hz, ³J_{H-H}=5.5 Hz, 2H), 4.51 (dt, ²J_{H-F}=47.0 Hz, ³J_{H-H}=4.5 Hz, 2H), 5.27 (bs, 2H), 7.56 (dd, ³J_{H-H}=8.5 Hz, ⁴J_{H-H}=1.0 Hz, 1H), 7.80-7.85 (m, 5H), 7.95 (ddd, ³J_{H-H}=7.5 Hz, ³J_{H-H}=7.0 Hz, ⁴J_{H-H}=1.0 Hz, 1H), 8.15-8.25 (m, 2H), 8.38 (ddd, ³J_{H-H}=8.0 Hz, ³J_{H-H}=7.0 Hz, ⁴J_{H-H}=1.0 Hz, 1H), 8.73 (d, ³J_{H-H}=8.0 Hz, 1H), 9.26 (d, ³J_{H-H}=8.5 Hz, 1H), 9.32 (dd, ³J_{H-H}=8.0 Hz, ⁴J_{H-H}=1.5 Hz, 1H).

¹³C NMR ((CD₃)₂SO, 125 MHz): δ 29.7 (d, ³J_{C-F}=19.6 Hz), 51.3 (d, ³J_{C-F}=4.9 Hz), 81.0 (d, ¹J_{C-F}=162.3 Hz), 120.67, 120.7 (q, ¹J_{C-F}=320.25 Hz), 123.2, 124.5, 125.6, 126.0, 128.3, 129.3, 130.3, 130.4, 130.9, 131.4, 132.4, 132.6, 133.8, 134.5, 137.4, 164.4.

¹⁹F NMR ((CD₃)₂SO, 470 MHz): δ −77.7 (s, 3F), −220.9 (tt, ²J_{F-H}=47.1, ³J_{F-H}=27.3, 1F).

### Example 23. 3,6-Bis(dimethylamino)-10-(3-fluoropropyl)acridinium trifluoromethanesulfonate

3-Fluoropropropyl trifluoromethanesulfonate (0.167 g, 0.79 mmol) is added to a 50 ml round-bottom single-neck flask with a magnetic dipole. 12 ml DCM is added using a needle and syringe. The solution is cooled to a temperature of −56° C. using a cooling bath (liquid nitrogen+ethyl acetate). A solution of Acridinium Orange (0.168 g, 0.63 mmol) in 12 ml DCM is added dropwise to the cooled solution over about 20 minutes, after which the contents of the flask are stirred for 72 h. At the end of the reaction, the reaction mixture is concentrated to about 12 ml, and 12 ml of cold Et₂O is added. The mixture is left in the refrigerator for 30 min, after which the product is filtered under reduced pressure. 0.339 g of the title compound is obtained (89.7% yield).

¹H NMR ((CD₃)₂SO, 500 MHz): δ 2.20 (d, ³J_{H-F}=28.0, 2H), 3.19 (s, 12H), 4.50-4.80 (m, 4H), 6.48 (s, 2H), 7.12 (d, ³J_{H-H}=9 Hz, 2H), 7.75 (d, ³J_{H-H}=9 Hz, 2H), 8.57 (s, 1H).

¹³C NMR ((CD₃)₂SO, 125 MHz): δ 26.7 (d, ²J_{C-F}=19.5 Hz), 40.1, 43.2, 81.7 (d, ¹J_{C-F}=161.3 Hz), 92.0, 114.0, 116.2, 120.7 (q, ¹J_{C-F}=320 Hz), 132.8, 142.0, 142.7, 155.2.

¹⁹F NMR ((CD₃)₂SO, 470 MHz): δ −77.7 (s, 3F), −219.0 (tt, ²J_{F-H}=49.4 Hz, ³J_{H-H}=25.1 Hz, 1F).

### Example 24. 3,6-Bis(dimethylamino)-10-(6-trifluoromethylsulfonyloxyhexyl)acridinium trifluoromethanesulfonate

1,16-Bis(trifluoromethanesulfonyloxy)hexane (0.434 g, 1.14 mmol) is added to a 50 ml round-bottom single-neck flask with a magnetic dipole. 6 ml DCM is added using a needle and syringe. The solution is cooled to a temperature of −56° C. using a cooling bath (liquid nitrogen+ethyl acetate), and a solution of Acridinium Orange (0.285 g, 1.07 mmol) in 12 ml DCM is added dropwise to the cooled solution over about 20 minutes. The contents of the flask are stirred for 96 h, and at the end of the reaction, 20 ml of cold Et₂O is added. The mixture is left in the refrigerator for 30 min, after which the precipitate formed is filtered off under reduced pressure to obtain 0.370 g of raw product with an estimated content of the title compound of approx. 33% (as determined based on signal integration in the ¹H NMR spectrum).

¹H NMR ((CD₃)₂SO, 500 MHz): δ 1.0-2.0 (m, 8H), 3.22 (s, 12H), 4.40 (bs, 2H), 4.53 (bs, 2H), 6.28 (s, 2H), 7.02 (d, J=7.5 Hz, 2H), 7.65 (d, J=8.0 Hz, 2H), 8.43 (s, 1H).

¹³C NMR ((CD₃)₂SO, 125 MHz): δ 22.7, 25.0, 29.1, 29.2, 32.4, 46.6, 69.6, 91.8, 113.9, 114.2, 116.4, 132.7, 132.8, 142.4, 155.2.

¹⁹F NMR ((CD₃)₂SO, 470 MHz): δ −77.8 (s, 3F).

### Example 25. The Method of Manufacturing ¹⁸F-Radiolabelled Compounds According to the Disclosure Using the Method Illustrated by the Reaction Sequence According to Scheme I

¹⁸F-labelled derivatives are synthesised using the Modular Lab Standard (Eckert&Ziegler) synthesiser. The ¹⁸F radioisotope is produced using Siemens Eclipse cyclotron in the ¹⁸O(p,n)¹⁸F reaction. The resulting ¹⁸F is deposited on the anion-exchange column QMA, from which water enriched with H₂¹⁸O is recovered. The fluoride compound ¹⁸F⁻ is eluted from the QMA column to the reactor using 600 μl kryptofix solution (22 mg) with potassium carbonate (11.7 mg) using H₂O:ACN eluent (1:1). Solvents are distilled off under reduced pressure, and then residual water is removed through azeotropic distillation by adding 1 ml ACN twice. A precursor, i.e. a quaternary ammonium salt of a polycyclic aromatic amine having a leaving group (preferably a trifluoromethanesulfonate group) in the chain side substituent on the quaternary nitrogen atom (in the amount of 12 to 20 mg) in DCM, is added to the reactor. The reaction is carried out at a temperature of 90° C. for 6.5 to 10 min. The reactor is cooled, and the solution is transferred to a collection vial. The reactor is then washed with a solution of acetate buffer, pH=5.2 supplemented with ethanol (12 to 28.5%), which is also transferred to the collection vial. The collected solution is applied on a semi-preparative HPLC column (250×10 mm, Luna, Phenomenex), using the acetate buffer, pH=5.2, mixed with ethanol as the mobile phase. For the phenanthridine derivative a mobile phase with 12.0% ethanol content is used, for the phenylphenanthridine derivative a mobile phase with 23.5% ethanol content is used, and for the acridine derivative a mobile phase with 28.5% ethanol content is used.

Based on the tests carried out with the use of standards, a product was obtained through collection of fractions from the semi-preparative column with a specified retention time. For the phenanthridine derivative, the product is collected directly into the final vial, while the acridine and phenylphenanthridine derivatives are transferred to a second reactor to remove excess ethanol through distillation at a temperature of 105-110° C. for 8 to 10 min followed by transferring the product to the final vial. The labelling yield under these conditions is 1-5%.

The radioisotope-labelled compound according to the disclosure obtained in the form of acetate salt is analysed by high-performance liquid chromatography (HPLC) to confirm the identity (by comparison with the standard provided by a structural analogue of the radioisotope-labelled compound of the quaternary ammonium salt of a polycyclic aromatic amine according to the disclosure, wherein said analogue has a non-radioactive fluorine atom, i.e. ¹⁹F, replacing ¹⁸F at the corresponding position at R² substituent carbon atom) and to determine the level of chemical and/or radiochemical contaminants, if any. The identity of the ¹⁸F isotope is confirmed by determining half-life (110 min) and measuring the gamma radiation energy of the ¹⁸F isotope (main peak of 511 KeV). In addition, thin-layer chromatography (TLC) is used to determine radiochemical purity.

The method for manufacturing ¹⁸F-radiolabelled compounds according to the disclosure using the method as above also allows for obtaining the product in the form of a salt with a counterion other than the acetate anion. For this purpose, experiments were carried out using a semi-preparative HPLC column (250×10 mm, Luna, Phenomenex) used to obtain ¹⁸F-labelled compounds, on which column N-(2-fluoroethyl)-6-phenylphenanthridinium trifluoromethanesulfonate was loaded. Mixtures of acetonitrile and three different buffers of 0.1 M phosphoric acid (pH=2.4), ascorbic acid (pH=6.3) or citric acid (pH=6.3) concentration, respectively, in isocratic system, were used as eluents. Product samples obtained following the elution from the semi-preparative column were analysed by high resolution mass spectrometry to determine the structure of both the cation and anion of the eluted quaternary amine compound in the form of salt. The results of the analyses are summarised in table 1 below.

TABLE 1

| Buffer | Phosphate | Citrate | Ascorbate |
|---|---|---|---|
| pH | 2.4 | 6.3 | 6.3 |
| Phase composition | 25:75 | 27:73 | 27:73 |
| (ACN %:buffer %) | | | |
| Theoretical mass of the | 302.13395 | | |
| cation | | | |
| MS cation mass | 302.13381 | 302.13381 | 302.13377 |
| Anion | H₂PO₄_{^{−}} | C₆H₇O₇_{^{−}} | C₆H₇O₆_{^{−}} |
| Theoretical mass of the | 96.96852 | 191.01863 | 175.02371 |
| anion | | | |
| MS anion mass | 96.96807 | 191.01870 | 175.02369 |

The results obtained clearly demonstrate that depending on the buffer added to the eluent used, the required counterion salt is obtained, and in these specific experiments a compound of quaternary ammonium salt of a polycyclic aromatic amine is obtained, wherein the ammonium cation is accompanied by a dihydrogen phosphate, citrate or ascorbate anion, respectively.

### Example 26. Manufacture of a Pharmaceutical Form of the ¹⁸F-Radiolabelled Compound According to Disclosure

The synthesis, formulation and dispensing of the preparation are performed in hot chambers. The active ingredient, i.e. the radioisotope-labelled compound of a polycyclic quaternary aromatic amine according to the disclosure, is manufactured and purified in accordance with the procedure presented in the examples above, after which the appropriate pH of the active ingredient solution is determined using a buffer, e.g. citrate, acetate, phosphate buffer (optionally, if not necessary, no buffer is used). If required, a pharmacologically non-aqueous acceptable diluent/solvent is added to the solution (for example to improve solubility or stability, such as ethanol); optionally, the solution is not diluted. The resulting solution is added through a sterilisation filter, for example 0.22 μm in size, to a collection vial placed in a grade A purity isolator, where the final formulation and determination of the desired radioactive concentration is performed by way of dilution (if necessary) of the raw product with a physiological solution of sodium chloride or injection water. The resulting product in bulk is automatically distributed into the final containers (vials, or directly to syringes) through a 0.22 μm final sterilisation filter. Optionally, thermal final sterilisation is used where the thermal treatment does not adversely affect the stability of the product. The vials are placed in external protective shielding.

### Example 27. PET-CT Scan Performed Using an Animal Model

Male rats weighing approx. 250 g are quarantined for a period of no less than 5 days. The animals are anaesthetised in an induction chamber using a 3.5-4% isoflurane (Baxter AErrane) atmosphere. The anaesthetised animal is transferred under an anaesthesis-maintaining mask (1.5-2% isoflurane in the air). A catheter is mounted on the lateral caudal vein of the animal. A heparin solution (approx. 50 μl) is injected into the vein in order to check the patency of the catheter and prevent excessive blood clotting.

The animal is transferred to a measurement bed placed in a PET/SPECT/CT scanner (Albira Carestream) equipped with a sensor allowing for monitoring the number of breaths and a system providing anaesthesia during the measurement. The number of breaths during the measurement is regulated by the concentration of the isoflurane supplied in the air and maintained within the range of 50-70 breaths per minute. For the duration of the measurement, the eyes of the animal are protected with a protective preparation (Vidisic).

The animal is administered 100 to 200 μl of the ¹⁸F-labelled radiopharmaceutical of the compound according to the disclosure (a cardiotracer) and 100 to 200 μl saline solution (in order to transport as much cardiotracer as possible from the catheter's dead space).

When the tracer administration is over, the dynamic PET acquisition starts.

The PET acquisition takes 35 to 90 minutes, depending on the recommendations of the person ordering the scan. The acquisition consists of a sequence of scans with a duration of 30 to 500 s (shorter scans occur in the first phase of the acquisition due to the higher dynamics of changes in the tracer biodistribution).

After the PET acquisition is over, the test object is moved to the CT module, where the whole body scan is performed (45 kVp tube voltage, 400 mA current, 400 projections per rotation, 4 rotations).

After the acquisition is over, the animal is awakened from anaesthesia and placed in a cage for 48 h in order to observe whether the tracer administration induced any adverse effects. It is then euthanized or may be used for another experiment, if planned within the next few days.

The acquisition results are reconstructed into 3D images using the Albira Suite Reconstructor software. An analysis is performed to obtain the specific uptake value (SUV) of the cardiotracer for selected organs and tissues: heart muscle, cardiac blood pool, lungs, liver, kidneys, bladder.

FIG. 1-3 illustrate representative PET images obtained in the above experimental animal studies: summed (1) and cross-sectional (2-4) in the frontal (1-2), sagittal (3) and transverse (4) plane of radioactivity distribution following the administration of compounds according to the subject disclosure to animals. More specifically, imaging of FIG. 1 was obtained by administering the pharmaceutical form of 5-(2-[¹⁸F]fluoroethyl)phenanthridinium salt, imaging of FIG. 2 was obtained by administering the pharmaceutical form of 6-phenyl-5-(2-[¹⁸F]fluoroethyl)phenanthridinium salt, and imaging of FIG. 3 was obtained by administering the pharmaceutical form of 3,6-bis(dimethylamino)-10-(2-[¹⁸F]fluoroethyl)acridinium salt. The pharmaceutical forms provided in the study were solutions of acetate salts in saline aqueous solution (i.e. cations of the ammonium salts of a polycyclic aromatic amine with formula I according to the disclosure referred to above were accompanied by chloride and acetate anions). The images are presented as the sum of time frames: 2-5 min (A) and 20-35 min (B).

### Example 28. Description of the Medical Procedure in Humans

Patients are placed on their back, with their hands behind their head, in the PET-CT scanner, and an intravenous catheter is placed in the lower part of the upper limb. Imaging begins with a resting scan in a dynamic data collection mode with the injection of a ¹⁸F-labelled radiopharmaceutical according to the disclosure. In the next scan, imaging is performed under stress following a pharmacological or physical stress. For dynamic imaging, the data acquisition start time is a few seconds before tracer administration. Perfusion images are acquired for 10 minutes directly following the intravenous tracer bolus administration. The minimum interval between the scan at rest and under stress is 50 minutes. Stress is induced in the patient through physical exercise or pharmacologically by administering regadenosone intravenous bolus (0.4 mg) regardless of body weight for 20-30 s. For regadenosone, the cannula is flushed with 5 ml saline directly after the administration, followed by the administration of a radioisotope-labelled compound according to the disclosure labelled with ¹⁸F (cardiotracer) 30 seconds after the saline. The PET scan is carried out as follows: 10 minutes of dynamic scan (12×10 seconds, 4×30 seconds, 1×6 minutes) in the area of the heart with an additional area above and below; 3D data acquisition, ECG gating (8 or 16 frames per cycle); array: 128×128.

PET imaging is performed at rest and under stress using PET-CT tomographs. With CT scans, attenuation adjustment is achieved 2 minutes before or after the test at rest and under stress. Low-dose CT is performed within 3 minutes before or after the acquisition of dynamic scans. The total CT scan time is 20 seconds. The rotation time of the X-ray tube is 0.5 second at 140 kV and 30 mA. Details of the myocardial perfusion imaging are summarised in table 2.

TABLE 2
Rest and stress myocardial perfusion imaging

| Characteristic | Imaging parameters |
|---|---|
| Stress conditions | Pharmacological agent regadenoson or physical |
| | strain |
| Tracer dose (3D) | (2-12 MBq/kg) usually 3-4 MBq/kg |
| Delay for static images | 1.5-3 minutes after the administration |
| Delay for dynamic | Activating the device directly before injecting |
| images | the tracer dose |
| PET/CT | CT scout |
| Imaging mode | ECG-gated imaging of perfusion and functional |
| | parameters of the myocardium |
| Mode: gated/dynamic | |
| Imaging duration | 12-15 minutes |
| Attenuation adjustment | Attenuation adjustment measurement before or |
| | after the acquisition |
| Reconstruction method | Iterative expectation maximisation method (e.g. |
| | OSEM) |
| Reconstruction filters | Sufficient to achieve the desired |
| | resolution/smoothing, matching stress and rest |
| | conditions |
| Reconstructed voxel size | 3.27 |

1. A radioisotope-labelled compound comprising a quaternary ammonium salt of a polycyclic aromatic amine having a structure according to formula I, 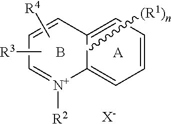
wherein a wavy line indicates a single bond between a non-nodal carbon atom of a polycyclic aromatic system and an R¹ substituent,
wherein the R¹ substituent selected from: a hydrogen; a halogen; a hydroxy; a protected hydroxy; a C₁₋₄ alkoxy; a nitro group; an amino group; an amino group having 1 hydrogen replaced with a C₁-C₆ alkyl group; an amino group having 2 hydrogens replaced with a C₁-C₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl;
wherein R² is a chain aliphatic substituent having:
a total of 1-16 carbon atoms,
an atom of ¹⁸F fluorine radioisotope replacing a hydrogen atom at one of the carbon atoms, and
a —CH₂ fragment as a terminal member of a chain, wherein the chain connects to one of a hydrogen, a phenyl group, and a phenyl group having 1-3 substituents selected from halogens and C₁₋₆ alkyl, and wherein if the chain contains at least 2 carbon atoms and there is a bivalent link between the chain carbon atoms, then the bivalent link is selected from the group consisting of an oxygen atom —O—, a sulfur atom —S—, and a C₃₋₆ cycloalkylene;
wherein R³ and R⁴ are combined to form a bivalent butadienyl-1,3 substituent whose terminal carbon atoms are linked to adjacent non-nodal carbon atoms of a B ring to form an aromatic C ring fused with an A and B ring system, having R¹ substituents at non-nodal carbon atoms;
wherein n is an integer of 9;
wherein X⁻ is a pharmaceutically acceptable counter ion selected from: an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

2. The radioisotope-labelled compound according to claim 1,
wherein the R¹ substituent is selected from: the hydrogen; the halogen; the C₁₋₄ alkoxy; the nitro group; the amino group; an amino group having 1 hydrogen replaced with a C₁-C₄ alkyl group; an amino group having 2 hydrogens replaced with the C₁-C₄ alkyl group; a chain C₁₋₄ carbon group; a chain C₁₋₄ carbon group having a substituent selected from the halogen and a C₁₋₄ alkanesulfonic; a phenyl group; a phenyl group having 1-3 substituents independently selected from halogens, a chain C₁₋₄ carbon, a halogenated chain C₁₋₄ carbon substituent, the C₁₋₄ alkoxy, and the amino group having 1-2 atoms of hydrogen replaced with C₁₋₄ alkyl; and
wherein for R², if the chain contains at least 2 carbon atoms and there is a bivalent link between the chain carbon atoms, then the link is selected from the group consisting of the oxygen atom —O— and the sulfur atom —S—.

3. The radioisotope-labelled compound according to claim 2,
wherein the R¹ substituent is selected from: the hydrogen; the halogen; the C₁₋₄ alkoxy; the amino group; the amino group having 1 hydrogen replaced with the C₁-C₄ alkyl group; the amino group having 2 hydrogens replaced with the C₁-C₄ alkyl group; the chain C₁₋₄ carbon group; the chain C₁₋₄ carbon group having a substituent selected from the halogen, the phenyl group; the phenyl group having 1-3 substituents independently selected from halogens, the chain C₁₋₄ carbon, the halogenated chain C₁₋₄ carbon substituent, and the C₁₋₄ alkoxy; and
wherein for R², if the chain contains at least 2 carbon atoms and there is a bivalent link between the chain carbon atoms, then the link is the oxygen atom —O—.

4. The radioisotope-labelled compound according to claim 3,
wherein R¹ substituent is selected from: the hydrogen; the halogen; the C₁₋₄ alkoxy; the amino group; the amino group having 1 hydrogen replaced with a C₁-C₂ alkyl group; the amino group having 2 hydrogens replaced with the C₁-C₂ alkyl group; the chain C₁₋₄ carbon group; the chain C₁₋₄ carbon group having a substituent selected from the halogen, the phenyl group; the phenyl group having 1-3 substituents independently selected from halogens, the chain C₁₋₄ carbon, and the halogenated chain C₁₋₄ carbon substituent; and
wherein for R², the bivalent link between the chain carbon atoms is absent, and the chain connects to one of a hydrogen, a phenyl group, and a phenyl group having 1-3 substituents selected from halogens and C₁₋₄ alkyl.

5. The radioisotope-labelled compound according to claim 4,
wherein the R¹ substituent is selected from: the hydrogen; the halogen; the amino group; the amino group having 1 hydrogen replaced with the C₁-C₂ alkyl group; the amino group having 2 hydrogens replaced with the C₁-C₂ alkyl group; the chain C₁₋₄ carbon group; the phenyl group; the phenyl group having 1-3 substituents independently selected from halogens, the chain C₁₋₄ carbon, and the halogenated chain C₁₋₄ carbon substituent
wherein for R², the chain connects to one of a hydrogen, a phenyl group, and a phenyl group having 1-3 substituents selected from the halogens.

6. A positron emission tomography diagnostic method, the method comprising:
(a) administering a radioisotope-labelled compound to a subject; and
(b) performing a positron emission tomography scan on the subject,
wherein the radioisotope-labelled compound comprises:
the radioisotope-labelled compound comprising a quaternary ammonium salt of a polycyclic aromatic amine having a structure according to formula I: 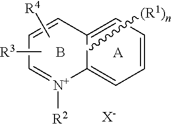
wherein a wavy line indicates a single bond between the non-nodal carbon atom of a polycyclic aromatic system and an R¹ substituent,
wherein the R¹ is a substituent selected from: a hydrogen; a halogen; a hydroxyl; a protected hydroxyl; a C₁₋₄ alkoxy; a nitro group; an amino group having 1 hydrogen atom replaced with C₁₋₆ alkyl group; an amino group having 2 hydrogen atoms replaced with C₁₋₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, a carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl;
wherein R² is a chain aliphatic substituent having:
a total of 1-16 carbon atoms,
an atom of ¹⁸F fluorine radioisotope replacing a hydrogen atom at one of the carbon atoms, and
a —CH₂ fragment as a terminal member of a chain, wherein the chain connects to one of a hydrogen, a phenyl group, and a phenyl group having 1-3 substituents selected from halogens and C₁₋₆ alkyl, and wherein if the chain contains at least 2 carbon atoms and there is a bivalent link between the chain carbon atoms, then the bivalent link is selected from the group consisting of an oxygen atom —O—, a sulfur atom —S—, and a C₃₋₆ cycloalkylene;
wherein R³ and R⁴ are combined to form a bivalent butadienyl-1,3 substituent whose terminal carbon atoms are linked to adjacent non-nodal carbon atoms of a B ring to form an aromatic C ring fused with an A and B ring system, having R¹ substituents at non-nodal carbon atoms;
wherein n is an integer of 9;
wherein X⁻ is a pharmaceutically acceptable counter ion selected from: an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

7. The positron emission tomography diagnostic method according to claim 6, further comprising examining the cardiovascular system of the subject, and
wherein the subject is a mammal.

8. The positron emission tomography diagnostic method according to claim 6, further comprising testing a myocardial perfusion of a myocardium of the subject to quantify a regional blood flow.

9. The positron emission tomography diagnostic method according to claim 8, further comprising quantifying a coronary reserve of the subject.

10. A pharmaceutical composition comprising:
a pharmaceutically acceptable carrier or diluent; and
a radioisotope-labelled compound comprising a quaternary ammonium salt of a polycyclic aromatic amine having a structure according to formula I 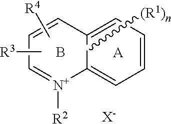
wherein a wavy line indicates a single bond between a non-nodal carbon atom of a polycyclic aromatic system and an R¹ substituent,
wherein the R¹ substituent is selected from: a hydrogen; a halogen; a hydroxy; a protected hydroxyl; C₁₋₄ alkoxy; a nitro group; an amino group having 1 hydrogen atom replaced with C₁₋₆ alkyl group; an amino group having 2 hydrogen atoms replaced with C₁₋₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl;
wherein R² is a chain aliphatic substituent having:
a total of 1-16 carbon atoms,
an atom of ¹⁸F fluorine radioisotope replacing a hydrogen atom at one of the carbon atoms, and
a —CH₂ fragment as a terminal member of a chain, wherein the chain connects to one of a hydrogen, a phenyl group, and a phenyl group having 1-3 substituents selected from halogens and C₁₋₆ alkyl, and wherein if the chain contains at least 2 carbon atoms and there is a bivalent link between the chain carbon atoms, then the bivalent link is selected from the group consisting of an oxygen atom —O—, a sulfur atom —S—, and a C₃₋₆ cycloalkylene;
wherein R³ and R⁴ are combined to form a bivalent butadienyl-1,3 substituent whose terminal carbon atoms are linked to adjacent non-nodal carbon atoms of a B ring to form an aromatic C ring fused with on A and B ring system, having R¹ substituents at non-nodal carbon atoms,
wherein n is an integer of 9;
wherein X⁻ is a pharmaceutically acceptable counter ion selected from: an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

11. The pharmaceutical composition according to claim 10, wherein the pharmaceutical composition is formulated as a sterile solution.

12. The pharmaceutical composition according to claim 10,
wherein the R¹ substituent is selected from: the hydrogen; the halogen; the C₁₋₄ alkoxy; the nitro group; the amino group; an amino group having 1 hydrogen replaced with a C₁-C₄ alkyl group; an amino group having 2 hydrogens replaced with the C₁-C₄ alkyl group; a chain C₁₋₄ carbon group; a chain C₁₋₄ carbon group having a substituent selected from the halogen and a C₁₋₄ alkanesulfonic; a phenyl group; a phenyl group having 1-3 substituents independently selected from halogens, a chain C₁₋₄ carbon, a halogenated chain C₁₋₄ carbon substituent, the C₁₋₄ alkoxy, and the amino group having 1-2 atoms of hydrogen replaced with C₁₋₄ alkyl; and
wherein for R², if the chain contains at least 2 carbon atoms and there is a bivalent link between the chain carbon atoms, then the link is selected from the group consisting of the oxygen atom —O— and the sulfur atom —S—.

13. The pharmaceutical composition according to claim 12,
wherein the R¹ substituent is selected from: the hydrogen; the halogen; the C₁₋₄ alkoxy; the amino group; the amino group having 1 hydrogen replaced with the C₁-C₄ alkyl group; the amino group having 2 hydrogens replaced with the C₁-C₄ alkyl group; the chain C₁₋₄ carbon group; the chain C₁₋₄ carbon group having a substituent selected from the halogen, the phenyl group; the phenyl group having 1-3 substituents independently selected from halogens, the chain C₁₋₄ carbon, the halogenated chain C₁₋₄ carbon substituent, and the C₁₋₄ alkoxy; and
wherein for R², if the chain contains at least 2 carbon atoms and there is the bivalent link between the chain carbon atoms, then the link is the oxygen atom —O—.

14. The pharmaceutical composition according to claim 13,
wherein R¹ substituent is selected from: the hydrogen; the halogen; the C₁₋₄ alkoxy; the amino group; an amino group having 1 hydrogen replaced with a C₁-C₂ alkyl group; an amino group having 2 hydrogens replaced with the C₁-C₂ alkyl group; the chain C₁₋₄ carbon group; the chain C₁₋₄ carbon group having a substituent selected from the halogen, the phenyl group; the phenyl group having 1-3 substituents independently selected from halogens, the chain C₁₋₄ carbon, and the halogenated chain C₁₋₄ carbon substituent; and
wherein for R², wherein for R², the bivalent link between the chain carbon atoms is absent, and the chain connects to one of a hydrogen, a phenyl group, and a phenyl group having 1-3 substituents selected from halogens and C₁₋₄ alkyl.

15. The pharmaceutical composition according to claim 14,
wherein the R¹ substituent is selected from: the hydrogen; the halogen; the amino group; the amino group having 1 hydrogen replaced with the C₁-C₂ alkyl group; the amino group having 2 hydrogens replaced with the C₁-C₂ alkyl group; the chain C₁₋₄ carbon group; the phenyl group; the phenyl group having 1-3 substituents independently selected from halogens, the chain C₁₋₄ carbon, and the halogenated chain C₁₋₄ carbon substituent,
wherein for R², the chain connects to one of a hydrogen, a phenyl group, and a phenyl group having 1-3 substituents selected from halogens.

16. A method for manufacturing a pharmaceutical composition, the method comprising combining a pharmaceutically acceptable carrier with a radioisotope-labelled compound, wherein the radioisotope-labelled compound comprises:
a quaternary ammonium salt of a polycyclic aromatic amine having a structure according to formula I: 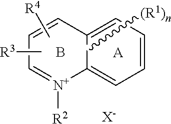
wherein a wavy line indicates a single bond between a non-nodal carbon atom of a polycyclic aromatic system and an R¹ substituent;
wherein the R¹ substituent is selected from: a hydrogen; a halogen; a hydroxy; a protected hydroxy; a C₁₋₄ alkoxy; a nitro group; an amino group; an amino group having 1 hydrogen replaced with a C₁-C₆ alkyl group; an amino group having 2 hydrogens replaced with a C₁-C₆ alkyl group; an amino group having 2 hydrogen atoms replaced with C₂₋₅ alkylene to form a heterocyclic ring; a chain C₁₋₆ carbon group; a chain C₁₋₆ carbon group having a substituent selected from a halogen, carboxyl, a formyl, and a C₁₋₄ alkanesulfonic; a phenyl group; a phenyl group having 1-5 substituents independently selected from halogens, a chain C₁₋₆ carbon, a halogenated chain C₁₋₆ carbon substituent, a hydroxy, a protected hydroxy, a C₁₋₄ alkoxy, and an amino group having 1-2 atoms of hydrogen replaced with C₁₋₆ alkyl;
wherein R² is a chain aliphatic substituent having:
a total of 1-16 carbon atoms,
an atom of ¹⁸F fluorine radioisotope replacing a hydrogen atom at one of the carbon atoms, and
a —CH₂ fragment as a terminal member of a chain, wherein the chain connects to one of a hydrogen, a phenyl group, and a phenyl group having 1-3 substituents selected from halogens and C₁₋₆ alkyl, and wherein if the chain contains at least 2 carbon atoms and there is a bivalent link between the chain carbon atoms, then the bivalent link is selected from the group consisting of an oxygen atom —O—, a sulfur atom —S—, and a C₃₋₆ cycloalkylene;
wherein R³ and R⁴ are combined to form a bivalent butadienyl-1,3 substituent whose terminal carbon atoms are linked to adjacent non-nodal carbon atoms of a B ring to form an aromatic C ring fused with an A and B ring system, having R¹ substituents at non-nodal carbon atoms;
wherein n is an integer of 9;
wherein X⁻ is a pharmaceutically acceptable counter ion selected from: an anion of a mono-basic inorganic acid, a mononegative anion of a multi-basic inorganic acid, an anion of an alkane carboxylic acid, an anion of an aliphatic sulfonic acid, an anion of an aromatic sulfonic acid, an anion of an acidic amino acid, a hydrate thereof, and a solvate thereof.

17. The method for manufacturing a pharmaceutical composition according to claim 16, further comprising sterilizing the pharmaceutical composition to form a sterile solution.
